(19) United States
(12) Patent Application Publication
(10) Pub. No.: US 2012/0302892 A1
(43) Pub. Date: Nov. 29, 2012

(54) PORTABLE OPTICAL FIBER PROBE-BASED SPECTROSCOPIC SCANNER FOR RAPID CANCER DIAGNOSIS

(76) Inventors: Niyom Lue, Nahant, MA (US); Michael Feld, Jamaica Plain, MA (US); Ishan Barman, Boston, MA (US); Narahara Chari Dingari, Somerville, MA (US); Ramachandra Dasari, Shererville, IN (US); David Feld, Newark, CA (US); Alison Hearn, Jamaica Plain, MA (US); Jonathan Feld, Somerville, MA (US)

(21) Appl. No.: 13/338,920

(22) Filed: Dec. 28, 2011

Related U.S. Application Data
(63) Continuation-in-part of application No. 11/492,301, filed on Jul. 25, 2006.
(60) Provisional application No. 60/702,248, filed on Jul. 25, 2005.
(60) Provisional application No. 61/569,095, filed on Dec. 9, 2011.

(51) Int. Cl. A61B 6/00

(52) U.S. Cl. 600/476

(57) ABSTRACT

A multimodal probe system for spectroscopic scanning of tissue for disease diagnosis. The system can use diffuse reflectance spectroscopy, fluorescence spectroscopy and Raman spectroscopy for the detection of cancerous tissue, such as tissue margin assessment.

### CROSS REFERENCE TO RELATED APPLICATION

This application claims the priority to U.S. application Ser. No. 11/492,301, filed Jul. 25, 2006, entitled MULTI MODAL SPECTROSCOPY, which claims priority to U.S. Provisional Application No. 60/702,248 filed Jul. 25, 2005 and further claims priority to U.S. Application No. 61/569,095 filed on Dec. 9, 2011. The entire contents of the above applications are being incorporated herein by reference.

### STATEMENT REGARDING FEDERALLY SPONSORED RESEARCH OR DEVELOPMENT

This invention was made with government support under Grant Nos. P41-RR02594 and R21-RR026259 awarded by the NIH National Center for Research Resources and Grant Nos. R01-CA97966 and R01-CA140288 from the National Cancer Institute. The government has certain rights in this invention.

### BACKGROUND OF THE INVENTION

Techniques capable of evaluating human disease in a safe, minimally invasive and reproducible way are of importance for clinical disease diagnosis, risk assessment, therapeutic decision-making, and evaluating the effects of therapy, and for investigations of disease pathogenesis and pathophysiology. Among the clinical methods available to diagnose tissue lesions, pathologic examination of cytology preparations, biopsies and surgical specimens is the present day standard.

Pathologists have traditionally based their diagnoses primarily on tissue morphology. However, as the field of diagnostic pathology has evolved, assessment of tissue morphology has become more sophisticated, including such techniques as morphometry (or quantitative image analysis) and ploidy analysis. Pathologic diagnosis has also begun to move from complete dependence on morphology to inclusion of a host of adjunct techniques that provide biochemical and molecular information as well. This is particularly true for the diagnosis of cancer, where routine diagnosis begins with morphology but usually also includes such molecular diagnostic techniques such as immunohistochemistry and in situ hybridization that identify specific molecular signatures.

This molecular information is not only of use for diagnosis, but is also of use for risk assessment and therapeutic decision-making, for example, in qualifying patients for molecular therapies, such as gene therapy or therapy with monoclonal antibodies directed against specific molecular targets. This molecular information has also greatly advanced the understanding of the pathogenesis and pathophysiology of many diseases, particularly cancer. But this evolution toward a focus on molecular events is not unique to the diagnosis of cancer. Recent molecular studies are also beginning to shed light on the pathogenesis and pathophysiology of cardiovascular disease, not only atherosclerosis but other disease (such as the cardiomyopathies) as well.

Intraoperative tissue diagnosis is an important component of successful cancer surgery in a variety of organ systems and tissues. Yet there continues to exist a significant clinical need for rapid and reliable intraoperative margin assessment of excised surgical specimens. Currently, intraoperative margin assessment is done by visual inspection and palpation, followed by selective assessment of any suspicious areas by rapid histology or cytology evaluation, which can be time consuming and inaccurate due to limited sampling. It is not unusual for the result of this pathologic margin assessment to come after the surgical wound is closed and the patient moved to the recovery room. Further, there are frequent discrepancies between the selective intraoperative and more comprehensive postoperative pathology margin assessment, necessitating a further operation to achieve negative margins. In breast-conserving surgery, for example, a second operation for positive margins discovered after surgery is required in up to 50% of cases. Furthermore, breast cancer recurs locally in the surgical site in −10% of patients with negative margins on postoperative pathology margin assessment which, while more comprehensive than intraoperative pathology assessment, is still subject to sampling limitations. Intraoperative assessment of surgical margins is an important aspect in surgical management of cancer.

### SUMMARY OF THE INVENTION

Diseases are more reliably identified by biochemical signatures than purely morphological markers. The present invention relates to the use of Raman spectroscopy in combination with other spectroscopic methods to provide biochemical and morphologic information and to further provide molecular information reflective of the metabolic state of tissue. This combination of biochemical, morphologic and metabolic information is used as the basis of more robust diagnostic methods. These types of molecular signature can be used for disease diagnosis, the disease progression and response to therapy.

Thus, in a preferred embodiment Raman and fluorescence can be used in combination to measure tissue in vivo using a probe or can be used to measure excised tissue samples. In a further embodiment Raman and reflected light can be used in combination for measurements on a human or animal body with a probe or on biological samples. Additionally, Raman, fluorescence and reflectance measurements can be made using a probe for in vivo or ex vivo measurements. A common light delivery and light collection probe can be used in preferred embodiments of the invention.

The combination of modalities in the modal spectroscopy (TMS) has several advantages over the single modalities alone. First, fluorescence spectroscopy provides information about tissue metabolites, such and NADH, that are not provided by Raman spectroscopy. Second, TMS uses diffuse reflectance spectroscopy (DRS) to overcome distortion of fluorescence signatures by the effects of tissue absorption and scattering, and extract the intrinsic fluorescence signature (IFS). Third, in addition to its value in extracting IFS, DRS provides critical information about the tissue absorbers and scatterers themselves. Finally, while DRS provides information about tissue components responsible for diffusive scattering, light scattering spectroscopy (LSS) provides information about tissue components responsible for single backscattering. The combination of techniques into TMS, therefore, provides a wealth of information about tissue fluorophores, absorbers and scatterers, which creates a much more complete biochemical, morphologic and metabolic tissue profile.

TMS and Raman methods have been applied to specific diseases based on the strengths of each spectroscopic modality for detecting the primary biochemical or morphologic hallmarks of that disease. For example, cancer is a characterized by rapid cellular proliferation that is reflected in increased cellular metabolism. TMS, which provides IFS and DRS information about key cellular metabolites such as NADH and oxy- and deoxy-hemoglobin is, thus, a natural choice of modality for the diagnosis of cancer. TMS also provides information about key morphologic cellular changes, such as the nuclear enlargement and pleomorphism (variation in size and shape), that are characteristic of cancer. On the other hand, vulnerable atherosclerotic plaque is the end result of an inflammatory process that leads to thinning and rupture of the fibrous cap, leading to the release of thrombogenic necrotic lipid core material into the blood stream. Atherosclerotic plaques are also subject to calcific mineralization of the fibrous cap and necrotic core. Most lipids and calcium salts are strong Raman scatterers and, thus, Raman spectroscopy is a natural choice of modality for the diagnosis of vulnerable atherosclerotic plaque.

The combination of spectroscopic modalities in multimodal spectroscopy (MMS) can provide information not provided by each modality. The whole (MMS) is also greater than the sum of the various individual modalities, because the biochemical and morphologic information provided is complementary—that is—the information provided by one technique often answers a question raised by the results of another. For example, for vulnerable atherosclerotic plaque, Raman spectroscopy provides information about the aggregate spectral contribution of foam cells and necrotic core, but raises questions about their individual contributions. Both DRS and light scattering answer those questions by providing specific information about the contribution of foam cells. So by combining the modalities in MMS one can decipher the separate contributions of both foam cells and necrotic core.

Measurements show that for vulnerable plaque, in some cases, two or more modalities are needed to fully characterize the contribution of a single tissue component. For example, as discussed above, oxy- and deoxy-hemoglobin are metabolites that may be key to the spectroscopic diagnosis of cancer. Hemoglobin is a strong tissue absorber and, therefore, it is a potential cause of distortion of tissue fluorescence signatures. This problem has been addressed in part by the use of TMS to derive undistorted IFS signatures. However, measurements in surgical breast biopsies have shown that in extremely bloody operative fields it is not be possible to account for all the absorbance effects of hemoglobin and achieve accurate diagnosis using TMS. On the other hand, hemoglobin is a weak Raman scatterer at NIR excitation wavelengths, and excellent model fits can be achieved for spectra acquired in bloody fields/tissues.

The combination of TMS and Raman spectroscopy in MMS provides a more complete and complementary biochemical, morphologic and metabolic tissue profiles than either TMS or Raman spectroscopy alone resulting in better diagnostic accuracy. Another key advantage in combining both techniques is the potential for depth sensing. TMS and Raman spectroscopy can use different excitation wavelengths, and therefore sample different tissue volumes because of wavelength-dependent differences in absorption and scattering. A Raman source preferably emits in a range of 750 nm to 1000 nm while the fluorescence source can employ one or more laser sources or a filtered white light source. Reflectance measurements preferably use a broadband source such as xenon flash lamp.

This difference in sampling volume can be exploited to provide information about the depth (or thickness) of certain tissue structures or layers. For example, the thickness of the fibrous cap is used for the diagnosis of vulnerable atherosclerotic plaque. The fibrous cap is composed largely of collagen. IFS and Raman spectroscopy both provide information about the contribution of collagen to tissue spectra. Comparison of the results from these two techniques, which use different excitation wavelengths and sample different tissue volumes, may provide information about the thickness of the fibrous cap. DRS and Raman spectroscopy both provide information about the contribution of deoxy-hemoglobin to the tissue spectra. Comparison of the results of these two techniques, which again use different excitation wavelengths and sample different tissue volumes, can provide depth-sensitive information useful in mapping cancers and pre-cancers of breast tissue.

Multimodal spectroscopy (MMS) is a system for spectral diagnosis and efficacy of combining spectroscopic results from TMS and Raman spectroscopy to provide better diagnostic detail and a more comprehensive picture of the biochemical, morphological and metabolic changes that occur in diseased tissues. The probe used in such measurements can be an endoscope or a small diameter probe for insertion through an endoscope channel or a small diameter catheter for insertion in the arterial system, for example.

The Raman methods for the diagnosis of breast cancer are based on a linear combination model similar to that used for peripheral arteries, that yields fit coefficients for epithelial cell nuclei and cell cytoplasm, fat cells, stromal collagen fibers, β-carotene, calcium oxalate and hydroxyapatite and cholesterol-like deposits (corresponding to tissue necrosis). The diagnostic procedure makes use of fit coefficients collagen and fat, two components of the tumor stroma.

Breast cancer, like most cancers, is characterized by abnormal cell proliferation and differentiation as well as increased cell metabolism. Autofluorescence, reflectance and LSS provide information about cell metabolism and tissue scatterers such as cell nuclei that is not provided by Raman spectroscopy. Therefore, by combining Raman spectroscopy with autofluorescence, reflectance and/or LSS, a method for the diagnosis of breast cancer incorporates contributions from both the malignant epithelial cells and the stroma.

Preferred embodiments of the invention relate to a portable, quantitative, optical fiber probe-based, spectroscopic tissue scanner designed for intraoperative diagnostic imaging of surgical margins. The tissue scanner combines diffuse reflectance spectroscopy (DRS) and intrinsic fluorescence spectroscopy (IFS), and has hyperspectral imaging capability, acquiring full DRS and IFS spectra for each scanned image pixel. A preferred embodiment can incorporate Raman detection into the probe used for scanning the region of interest. Modeling of the DRS and IFS spectra yields quantitative parameters that reflect the metabolic, biochemical and morphological state of tissue, which are translated into disease diagnosis. The tissue scanner has high spatial resolution (0.25 mm) over a wide field of view (10×10 cm), for example, and both high spectral resolution (2 nm) and high spectral contrast, readily distinguishing tissues with widely varying optical properties (bone, skeletal muscle, fat and connective tissue). Tissue-simulating phantom measurements confirm that the tissue scanner can quantitatively measure spectral parameters, such as hemoglobin concentration, in a physiologically relevant range with a high degree of accuracy (<5% error). Measurements using human breast tissues showed that the tissue scanner can detect small foci of breast cancer in a background of normal breast tissue. This tissue scanner is simpler in design, images a larger field of view at higher resolution and provides a more physically meaningful tissue diagnosis than existing spectroscopic imaging systems. This spectroscopic tissue scanner can provide real-time, comprehensive diagnostic imaging of surgical margins in excised tissues, overcoming the sampling limitation in current histopathology margin assessment. Preferred embodiments can use a fiber optic probe for manual scanning of surgical sites.

The challenge in addressing this need is to develop an imaging system with a wide enough field of view to image large surgical specimens with high enough resolution to detect small foci of cancer at the surgical margin in a clinically useful time frame. It is desirable, for example, to scan a tissue sample exceeding 10 cm² in area in less than 20 minutes to achieve this objective.

Optical fiber probe-based diffuse reflectance spectroscopy (DRS), intrinsic fluorescence spectroscopy (IFS) and Raman spectroscopy are used as tools for the real time diagnosis of cancer, and have advantages over other approaches to intraoperative and comprehensive assessment of surgical sites and margins. DRS, IFS and Raman measurements depend on the inherent optical properties of tissue and, as such, do not require exogenous imaging probes or contrast agents. The combination of DRS, IFS and Raman measurements provide information on the metabolic, biochemical and morphological state of tissue, which can be translated into disease diagnosis. DRS and IFS have relatively shallow (≦1 mm) tissue penetration, and thus interrogate only the margin of the excised tissue specimen. Unlike traditional pathology diagnosis, spectroscopic diagnosis can be performed in real time.

Spectroscopic techniques are also quantitative and therefore more objective than the traditional methods. Conventional optical fiber probe-based spectroscopy techniques only examine a small area of tissue (˜1 mm) at a time, and so can suffer from under sampling and easily miss the lesion of interest. Spectroscopic imaging techniques can examine the entire margin of the excised tissue specimen, and so are not prone to the sampling limitations inherent in traditional pathology examination.

A preferred embodiment of the present invention includes a portable, quantitative, optical fiber probe-based, spectroscopic tissue scanner that can provide real-time comprehensive assessment of surgical margins in excised tissue specimens. The scanner significantly extends existing optical fiber probe-based spectroscopy instruments, which can be employed in the diagnosis of oral, esophageal, cervical, skin and breast cancer but previously unavailable in a wide field, high resolution imaging system. This tissue scanner is simpler in design, images a larger field of view at higher resolution and provides a more physically meaningful tissue diagnosis. The tissue scanner can provide fast, accurate, diagnostic images of the entire margin of excised surgical specimens, overcoming the sampling limitation in current pathology margin assessment.

A preferred embodiment can employ an inverted geometry with the sample placed on a optically transparent support and scanned by providing relative movement between a fiber optic light delivery and collection system and the tissue sample. Either the tissue sample or the fiber optic probe can be scanned to achieve the desired scan area, resolution and scan time. These scan parameters can be selected based on the size and geometry of the tissue sample. Note that pressure can be applied uniformly across the tissue surface to provide contact with the scanned surface. This provides precise spot size and distance to the probe to achieve this required pixel by pixel registration of images.

### BRIEF DESCRIPTION OF THE DRAWINGS

FIG. 1A is a schematic illustration of an MMS system in accordance with a preferred embodiment of the invention;

FIG. 1B is a scatter plot of Raman data;

FIG. 2a-2b are basis spectra;

FIGS. 3A-3C are scatter plots of an MMS system;

FIGS. 4A-4C are plots of an MMS system;

FIGS. 5A and 5B shows Raman basis spectra;

FIG. 6A-6C show spectra and fits for MMS modes;

FIG. 7 is a bar graph for hemoglobin concentration;

FIG. 8 shows scattering parameter A for DRS;

FIG. 9 is a plot of the coefficients ratio for IFS;

FIG. 10 is a plot of the Raman parameter for artery samples;

FIG. 11A are graphs of coefficients for artery tissue;

FIGS. 11B-D are Raman, reflectance and fluorescence data of an artery;

FIG. 12 shows sampling depths;

FIGS. 13A and 13B include side and end views, respectively, of a Raman Probe;

FIG. 13C is a side cross-sectional view of a side looking probe;

FIG. 13D is an end view of an MMS probe in accordance with the invention;

FIG. 13E is a forward looking MMS probe with a ball lens;

FIG. 13F is a forward looking MMS probe with a half ball lens;

FIG. 13G graphically illustrates a distal filter system for an MMS probe;

FIG. 14A is a schematic of an MMS system;

FIG. 14B is another embodiment of an MMS system;

FIG. 15A is a schematic diagram of the tissue scanner in accordance with preferred embodiment of the inventors;

FIG. 15B is a cross-sectional view of a fiber optic probe used in conjunction with preferred embodiments of the invention;

FIG. 16A is an image of the resolution target and a randomly selected 2D 2.5 cm×2.5 cm DRS spectral intensity map of zoomed target at 510 nm;

FIG. 16B is a photograph of the porcine lower leg tissue cross section that was scanned;

FIG. 16C is an image of the tissue cross section on the glass plate during scanning;

FIGS. 16D-16H are 9 cm×9 cm DRS images of the tissue cross section at different wavelengths; note that the color bar is for all DRS images and is in arbitrary units;

FIGS. 17A-17C show fifteen liquid phantoms composed of 2% intralipid and varying Hb concentrations in glass vials; O-rings filled with liquid phantoms and spectralon standards (10% and 20%) on the glass plate, with the scanning field of view marked with yellow tape; 2D DRS scan of the phantoms and spectralon standards at 490 nm (field of view=10×10 cm), respectively;

FIGS. 18A and 18B include a DRS spectrum from a liquid phantom with 1.8 mg/ml Hb (blue), corresponding model fit (red) and residual (black) and a bar chart showing the Hb concentration curve results, respectively;

FIGS. 19A and 19B are DRS and IFS spectra, respectively, of phantoms with varying furan concentration (phantom 1 and 2 furan=0.8 μg/mL); phantom 3 and 4 furan=0.3 μg/mL);

FIGS. 20A-20E include a diagram of normal and breast cancer tissues placed on glass plate during scanning, a photograph of breast tissues, a composite photomicrograph of histopathology of breast tissues (top insert: ductal carcinoma in situ; insert: invasive ductal carcinoma); and DRS and IFS spectra of breast tissues, respectively;

FIGS. 21A-21H include a DRS spectral intensity map of normal and cancer tissues at 545 nm; IFS spectral intensity map at 425 nm; DRS parameter maps for scattering parameters A, B and C, Hb and β-carotene, respectively, and an IFS parameter map for collagen;

FIG. 22 is a bar chart of pixel-to-pixel means DRS and IFS parameters in normal and breast cancer tissues;

FIGS. 23A-23B include a scatterplot of decision algorithm using DRS β-carotene and IFS collagen parameters for selective regions in normal and cancer breast tissues and a diagnostic map of normal and breast cancer tissues using the decision algorithm, respectively, noting that: 1) regions denoted as cancer at the border of the grossly cancerous and normal tissues (white arrows) are an artifact due to inadvertent gaps between the two tissues; and 2) regions denoted as cancer at the outer edges of the tissues (black arrows) are due to the presence of black colloidal ink applied to the margin of the surgical specimen prior to harvesting for use;

FIGS. 24A and 24B illustrate reduced scattering and absorption coefficients as a function of wavelength, where the dashed line indicates the raw data and PLS, LUT and LS-SVM model fits are shown with green, blue and red solid lines, respectively. For this representative phantom, PLS (green) and LS-SVM (red) fits for the reduced scattering coefficient are nearly coincident. Similarly, the raw data (black) and LS-SVM (red) fit coalesce for the absorption coefficient plot;

FIGS. 25A and 25B are box plots of prediction error percentages for reduced scattering (μ_{s′}) and absorption (μₐ) coefficients, respectively, using PLS, LUT and LS-SVM regression models; the red dotted line indicates the position where the observed values are equal to the reference values in the samples; and

FIG. 26 is a bar plot of average computation time for the prediction step of PLS, LUT and LS-SVM algorithms.

### DETAILED DESCRIPTION OF THE INVENTION

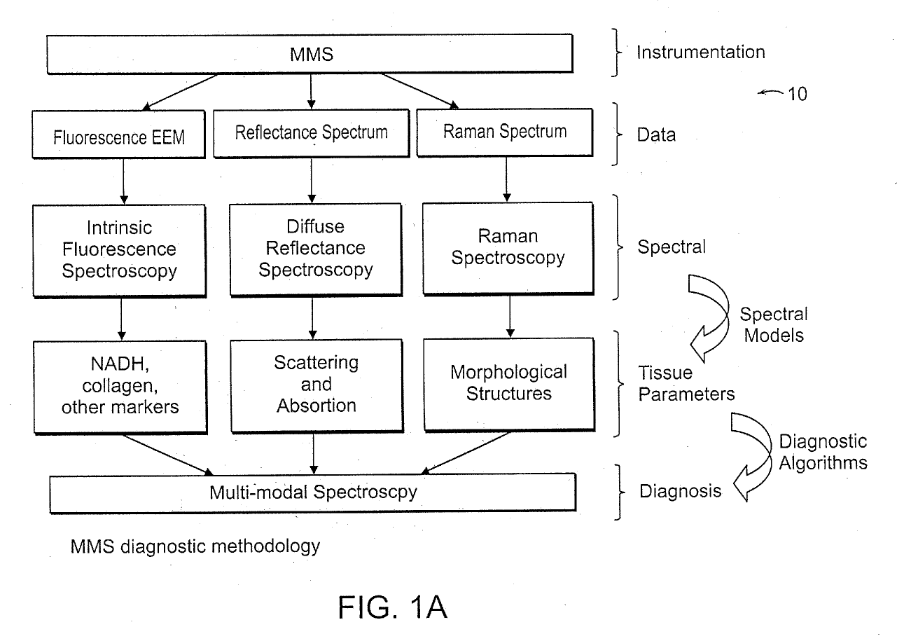

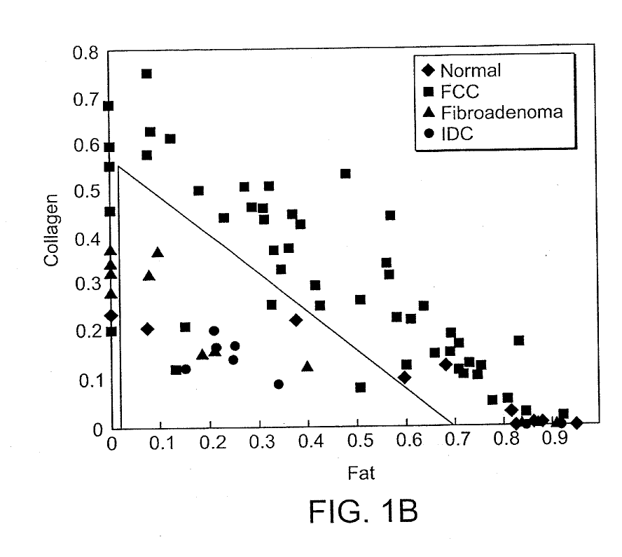

An MMS system is generally illustrated in FIG. 1A. MMS measurements have been performed on surgical biopsies within 30 minutes of surgical resection. Most of the 30 minute time delay was due to inking and sectioning of the specimen performed as part of the routine pathology consultation performed on these specimens for more information on intra-operative margin assessment in breast cancer patients. IFS, diffuse reflectance and Raman spectra were obtained from a total of 223 spectra from 105 breast tissues from 25 patients. Specimens from patients with pre-operative chemotherapy or who underwent re-excisional biopsy were excluded. DRS and IFS spectra were collected using the FastEEM instrument, followed by collection of Raman spectra with a Raman instrument. Care was taken in placing the Raman probe at the same site on the tissue as the FastEEM probe. Once the spectra were acquired, the exact spot of probe placement was marked with colloidal ink for registration with histopathology. The breast specimens were then fixed and submitted for routine pathology examination, which was performed by an pathologist blinded to the spectroscopy results. The histopathology diagnoses were: 32 normal; 55 fibrocystic change, 9 fibroadenoma and 9 invasive carcinoma (all infiltrating ductal carcinoma).

The sampled tissue volume for Raman spectroscopy is 1 mm³. Using the combined biochemical and morphologic spectral model, the data are fit to a linear combination of Raman basis spectra for eight breast tissue components: cell cytoplasm, cell nucleus, stromal collagen fibers, fat cells, β-carotene, collagen, calcium hydroxyapatite, calcium oxalate dehydrate, and cholesterol-like lipid deposits (foci of necrosis). The data were then analyzed prospectively using the fit coefficients for stromal collagen (collagen) and fat cells (Fat) in our Raman algorithm for breast cancer diagnosis. A scatter plot and decision lines for the Raman diagnostic algorithm are shown in FIG. 1B. A comparison of the Raman spectral diagnoses and histopathology diagnoses is shown in Table 1. The Raman algorithm remained quite robust when applied in a prospective manner to these breast specimens, with an overall accuracy of 83%. However, five cases of fibroadenoma were misdiagnosed by Raman as invasive cancer and 4 cases of fibrocystic change were misdiagnosed as cancer.

TABLE 1
Comparison of pathologic diagnosis with that of the Raman
diagnostic algorithm for ex-vivo specimens. The TMS diagnostic
algorithm resulted in an overall accuracy of 81% (85/105).

| | Pathology | | | |
|---|---|---|---|---|
| | Normal | Fibrocystic | | Invasive |
| | (32 | Change | Fibroadenoma | Carcinoma |
| Raman | samples) | (55 samples) | (9 samples) | (9 samples) |
| Normal | 30 | 7 | 0 | 0 |
| Fibrocystic | 2 | 41 | 0 | 0 |
| Change | | | | |
| Fibroadenoma | 0 | 3 | 4 | 1 |
| Invasive | 0 | 4 | 5 | 8 |
| Carcinoma | | | | |

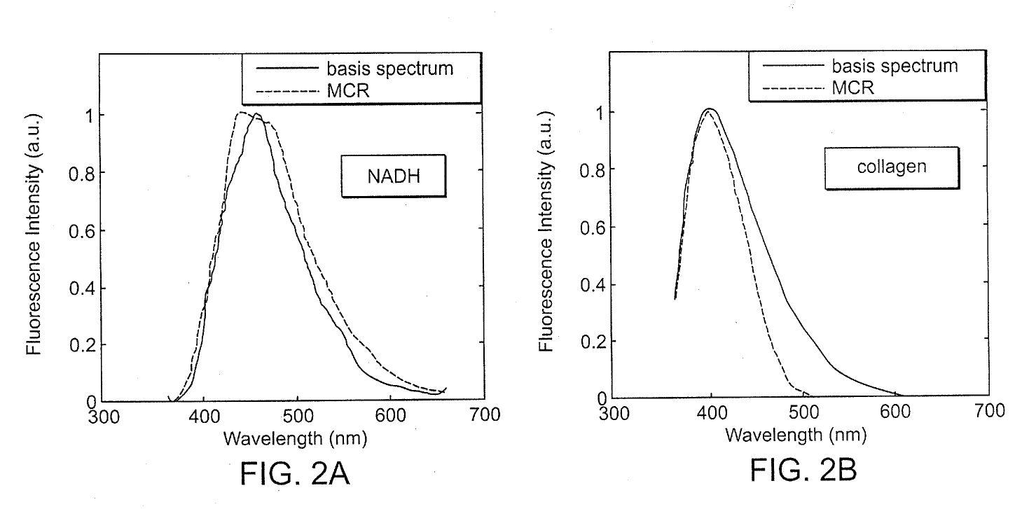

IFS were extracted from the combined fluorescence and DRS. The IFS spectra were analyzed using multivariate curve resolution (MCR) with non-negativity constraints, a standard chemometric method, to extract two spectral components at each excitation wavelength. The resulting MCR-generated spectral components at 340 nm are shown in FIG. 2a and FIG. 2b, and represent NADH and collagen, respectively, because they are similar to their measured IFS spectra. The spectra are similar, but not identical, as both the lineshape and wavelength maximum of a fluorescence peak obtained from a solution of a pure component is known to be different than that obtained from the same component in a different chemical environment, such as tissue.

For diffusive scattering (μₛ′), wavelength dependence of the form Aλ^{-B} is used. Two absorbers, oxyhemoglobin and β-carotene, were used to model the extracted absorption coefficient μₐ. Therefore, DRS provided, among other parameters, the amplitude of the scattering coefficient, A, and the concentration of oxyhemoglobin.

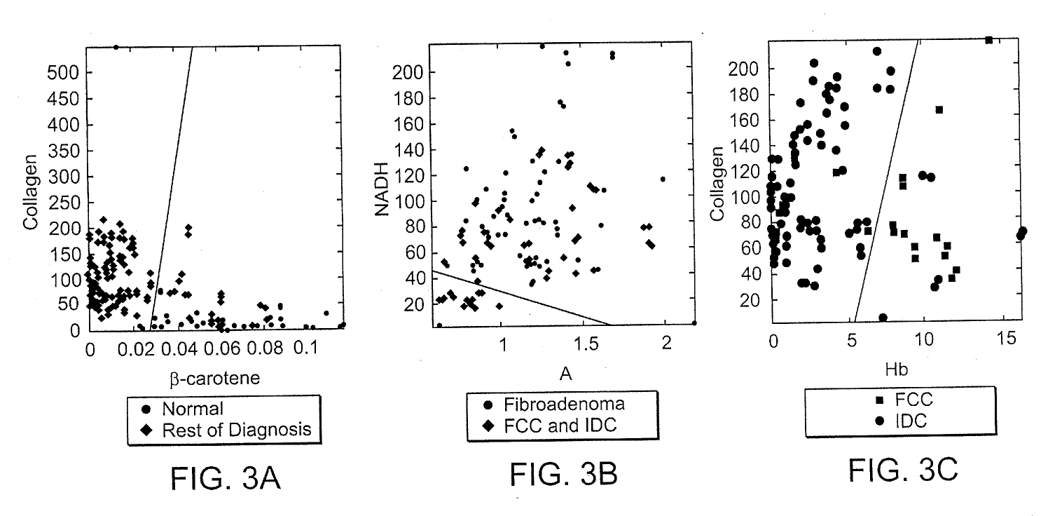

The TMS diagnostic method used logistic regression and leave-one-out cross validation, and the analysis was performed in sequential fashion. Scatter plots and decision lines for each step of the diagnostic method are shown in FIG. 3a-3c. Normal tissue was identified using the Raman fit coefficients for both collagen and β-carotene (FIG. 3a). The finding of low fit coefficients for collagen and β-carotene correlates with histopathology, as normal breast tissue consists largely of adipose tissue, the fat cells which contain large amount of lipid-soluble β-carotene. After the normal tissue was excluded, fibroadenoma was discriminated from fibrocystic change and invasive breast cancer, using the DRS scattering parameter A and IFS NADH fit coefficients (FIG. 3b). Fibrocystic change was distinguished from invasive breast cancer using the DRS oxyhemoglobin and IFS collagen fit coefficients at 340 nm (FIG. 3c). This diagnostic method uses contributions from both the cells (NADH) and the stroma (collagen). However, it is unclear why the fit coefficient for collagen and scattering parameter A should be lower for fibroadenoma than for invasive carcinoma and fibrocystic disease, or the fit coefficients for oxyhemoglobin should be higher for invasive breast cancer than for fibrocystic disease. A comparison of the TMS spectral diagnoses and histopathology diagnoses is shown in Table 2. The overall accuracy (correct prediction of each of the pathologies) is 87.6% (92/105). Although the overall accuracy of the two techniques is comparable in this small data set, all of the invasive carcinomas were diagnosed correctly by TMS and only 4 normals or fibrocystic changes were misclassified as invasive carcinoma.

TABLE 2
Comparison of TMS and histopathologic diagnosis for ex vivo
study of fresh surgical breast biopsies. The TMS diagnostic
algorithm had an overall accuracy of 87.6% (92/105).

| | Pathology | | | |
|---|---|---|---|---|
| | Normal | Fibrocystic | | Invasive |
| | (32 | Change | Fibroadenoma | Carcinoma |
| TMS | samples) | (55 samples) | (9 samples) | (9 samples) |
| Normal | 27 | 7 | 0 | 0 |
| Fibrocystic | 2 | 47 | 0 | 0 |
| Change | | | | |
| Fibroadenoma | 0 | 0 | 9 | 0 |
| Invasive | 3 | 1 | 0 | 9 |
| Carcinoma | | | | |

The measurements were obtained using TMS and Raman spectroscopic techniques independently, but they can also be obtained using a combined diagnostic procedure. In developing the MMS algorithm, only parameters that were diagnostic in one of the three individual spectroscopic modalities were used. The diagnostic parameters from TMS are scattering parameter A, and the fit coefficient for oxyhemoglobin, β-carotene, and NADH and collagen by IFS at 340 nm excitation wavelength. The diagnostic Raman parameters are the fit coefficients for fat and collagen. Like the TMS diagnostic procedure, this algorithm incorporates contributions from both the epithelial cells (NADH) and stroma (collagen).

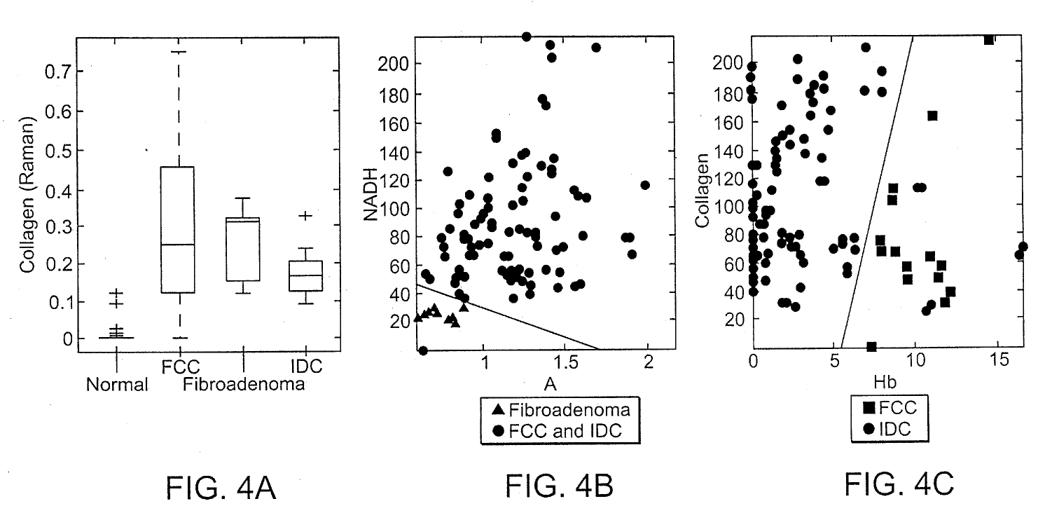

The MMS diagnostic method was developed using logistic regression and leave-one-out cross validation. As with TMS, the analysis is performed in sequential fashion. FIGS. 4A-4C display the scatter plots and decision lines for each of the three steps performed in the MMS diagnostic algorithm. First, normal tissue was identified using the Raman fit coefficient for collagen. This is the only change in this algorithm than that used for TMS, where the first step was identification of normal tissues using the intrinsic fluorescence fit coefficient for collagen at 340 nm (FIG. 4A). The next two steps are identical to those in the TMS diagnostic algorithm, with fibroadenoma distinguished from fibrocystic change and invasive carcinoma using scattering parameter A and the fit coefficient for NADH (FIG. 4b), and fibrocystic disease distinguished from invasive breast cancer using the fit coefficients for oxy-hemoglobin (FIG. 4C). A comparison of the MMS spectral diagnoses and histopathology diagnoses is shown in Table 3. The overall accuracy is 92% (92/105), and is only slightly improved for MMS as compared to TMS. As with TMS, all 9 invasive carcinomas were diagnosed correctly by MMS. But this time, only 2 fibrocystic changes and no fibroadenoma are diagnosed as invasive carcinoma.

TABLE 3
Comparison of MMS and histopathologic diagnosis for the ex vivo
study of surgical breast biopsies. The MMS diagnostic
algorithm had an overall accuracy of 92.4%.

| | Pathology | | | |
|---|---|---|---|---|
| | Normal | Fibrocystic | | Invasive |
| | (32 | Change | Fibroadenoma | Carcinoma |
| Multimodal | samples) | (55 samples) | (9 samples) | (9 samples) |
| Normal | 30 | 4 | 0 | 0 |
| Fibrocystic | 2 | 49 | 0 | 0 |
| Change | | | | |
| Fibroadenoma | 0 | 0 | 9 | 0 |
| Invasive | 0 | 2 | 0 | 9 |
| Carcinoma | | | | |

Table 4 shows a detailed comparison of the diagnostic performance of all three methods, Raman, TMS and MMS, with MMS providing the best sensitivity and specificity, as well as overall accuracy. By introducing a parameter from the Raman model to the first step a greater number of correctly diagnosed normal tissues. FIG. 4A is a box plot, which illustrates the average values (red line), the interquartile range (blue box), and outliers (red plusses), of collagen content for each pathology. Previously, the collagen content from TMS was analyzed in this manner but did not show the same success. Although both Raman and TMS (and thus MMS) are sensitive to collagen, each uses a different wavelength of light (Raman at 830 nm and TMS at 340 nm). Therefore, their sampling volumes are different. This fact explains why collagen fit coefficients extracted via Raman spectroscopy do not strongly correlate with collagen fit coefficients extracted using TMS. This is likely because of the different sample volumes (depths) of the TMS and Raman modalities. With a smaller sampling volume, TMS did appear to sample deep enough into the tissue to assess collagen adequately.

The results indicate that MMS, a combination of DRS, IFS, and Raman spectroscopy provides better results than those obtained from each technique alone. This can result from the combined MMS diagnostic algorithm combines spectral parameters derived from both epithelial cells and stroma and (taken together) have a larger sample volume.

TABLE 4
Comparison of performance of Raman, TMS and MMS
algorithms for the diagnosis of breast cancer.

| | Modality | | |
|---|---|---|---|
| Performance | Raman | TMS | MMS |
| Sensitivity | 89% | 100% | 100% |
| Specificity | 91% | 96% | 98% |
| Overall Accuracy | 81% | 88% | 92% |

As in breast cancer, the development of atherosclerosis is governed by subtle chemical and morphological changes in the arterial wall, manifesting themselves in the development of a plaque that causes luminal obstruction. Many of these changes are the result of metabolically active inflammatory and smooth muscle cells, such as foam cells, that degrade LDL and release it into the necrotic core in the form of ceroid and other LDL degradation byproducts.

The preferred method for the diagnosis of atherosclerosis are based on a linear combination model that yields fit coefficients for 10 morphological components of artery wall, including collagen fibers (CF), elastic lamina (EL), smooth muscle cells (SMC), adventitial adipocytes (AA), cholesterol crystals (CC), β-carotene crystals (β-CC), foam cells/necrotic core (FC/NC) and calcium mineralizations (CM). A preferred algorithm was developed for classification of lesions as non-atherosclerotic, non-calcified plaque and calcified plaque. This diagnostic algorithm was based on combined fit coefficients for cholesterol crystals+foam cells/necrotic core (the latter two having indistinguishable Raman basis spectra) and the fit coefficient for calcium mineralizations.

A preferred embodiment relates to a procedure for measuring vulnerable plaque. These are most often plaques with a thin fibrous cap overlying a large necrotic lipid core, and may have other features of vulnerability including foam cells and other inflammatory cells, intraplaque hemorrhage or thrombosis. A second Raman algorithm capable of diagnosing vulnerable plaque with about the same sensitivity and specificity as a previous algorithm for plaque classification (˜85-95%). This second algorithm for the diagnosis of vulnerable plaque makes use of the fit coefficients of 5 artery morphological components: the combined fit coefficients for foam cells+necrotic core and the fit coefficient for calcifications, as in the previous algorithm, plus the fit coefficients for collagen and hemoglobin. A preferred algorithm for the diagnosis of vulnerable plaque involves using spectral parameters that distinguish between metabolically active foam cells and the non-metabolically active necrotic core.

Fluorescence, reflectance and LSS provide information about cell metabolism and tissue scatterers such as foam cells, the cytoplasm of which is filled with a foam-like aggregate of lipid-filled lysosomal vesicles where the metabolism and degradation of LDL takes place. Therefore, by combining Raman spectroscopy with fluorescence, reflectance and optionally LSS, an algorithm for the diagnosis of vulnerable plaque incorporates contributions from metabolically active, potential scatterers like foam cells as well as non-metabolically active plaque constituents like the necrotic core. But, MMS has a further advantage for the diagnosis of vulnerable plaque, and that is the ability to provide depth information about key biochemical and morphologic structures like the fibrous cap, that too undergoes degradation, this time, by matrix metalloproteinase that renders it more prone to rupture.

In vitro measurements of MMS for the diagnosis of vulnerable plaque using 17 frozen archival tissues from carotid endarterectomies have been performed.

TMS spectra were collected using the FastEEM instrument and Raman using the clinical Raman system, with the associated probes. Care was taken in placing the Raman probe at the same site on the tissue as the FastEEM probe. Once the spectra were acquired, the exact spot of probe placement was marked with colloidal ink for registration with histopathology. The artery specimens were then fixed and submitted for routine pathology examination, which was performed by a cardiovascular pathologist blinded to the spectroscopy results. The histopathology examination of the lesions included an assessment of a number of histologic features of vulnerable plaque, including thickness of the fibrous cap, size of the necrotic core, superficial foam cells, intraplaque hemorrhage and ulceration. The histopathology results are summarized in Table 5. A vulnerable plaque index (VPI) was assigned to each specimen. Of the 17 lesions, 4 exhibited VPI scores ≧10 and were classified as vulnerable plaques.

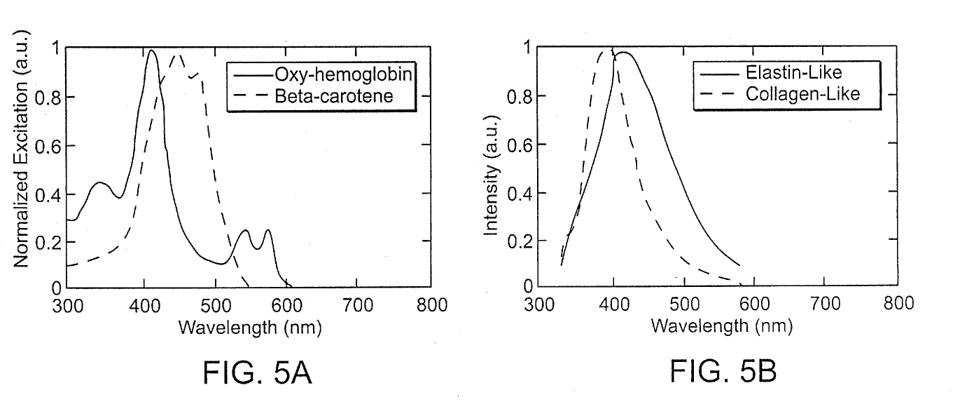

MMS spectral analysis for artery was similar to that for the breast. Again, OLS is used to fit the Raman data using the morphological model. The DRS spectra were fit using the diffusion theory model. Modeling of the DRS spectra yielded, among other parameters, scattering coefficient A and hemoglobin concentration. IFS were analyzed using MCR with non-negativity constraints to find two spectral components at 308 nm and 340 nm. The IFS data was fit using ordinary least squares (OLS) using the two MCR components as the model. The Raman basis spectra, DRS extinctions and IFS MCR components are shown in FIGS. 5A and 5B.

TABLE 5
Morphological characteristics of the 17 specimens.

| | | | Intimal or | Necrotic | | | | |
|---|---|---|---|---|---|---|---|---|
| | | | Fibrous cap | Core | Foam Cell | Foam Cell | | |
| | SNOMed | | Thickness | Thickness | Depth | Grade | Intraplaque | |
| | Class. | VPI | (microns) | (microns) | (microns) | (0-3+) | Hemorrhage | Ulceration |
| 1 | IF | 5 | 24-64 | NA | NA | NA | NA | NA |
| 2 | IF | 5 | 40-80 | NA | NA | NA | NA | NA |
| 3 | ATS | 0 | 480-500 | NA | 480 | 3+ | NA | NA |
| 4 | ATS | 5 | 240-440 | NA | 40 | 1+ | NA | NA |
| 5 | ATS | 0 | 456-536 | NA | 456 | 2+ | NA | NA |
| 6 | ATM | 5 | 200-320 | 400 | 280 | 2+ | NA | NA |
| 7 | ATM | 5 | 460-640 | 560 | NA | NA | NA | NA |
| 8 | ATM | 5 | 440-500 | 4800 | 440 | 2+ | NA | NA |
| 9 | ATM | 5 | 1000-1500 | 6400 | 1800 | 1+ | NA | NA |
| 10 | ATM | 5 | 520-640 | 1340 | 640 | 2+ | NA | NA |
| 11 | CATM | 5 | 140-160 | 1840 | 68 | 1+ | NA | NA |
| 12 | CATM | 7 | 120-480 | 4000 | 120 | 1+ | NA | NA |
| 13 | CATM | 5 | 1440-1600 | 240 | 256 | 1+ | NA | NA |
| 14 | CFS | 20 | 0-400 | NA | NA | NA | Acute | Fissure |
| 15 | FS | 15 | 40-80 | NA | NA | NA | NA | Rupture |
| 16 | ATS | 10 | 27-52 | NA | 0 | 1+ | NA | NA |
| 17 | ATM | 25 | 0-280 | 1600 | 28 | 2+ | NA | Rupture |

IF = infimal fibroplasias,
ATS = atherosclerotic,
ATM = atheromatous,
FS = fibrotic-sclerotic,
C = calcified.

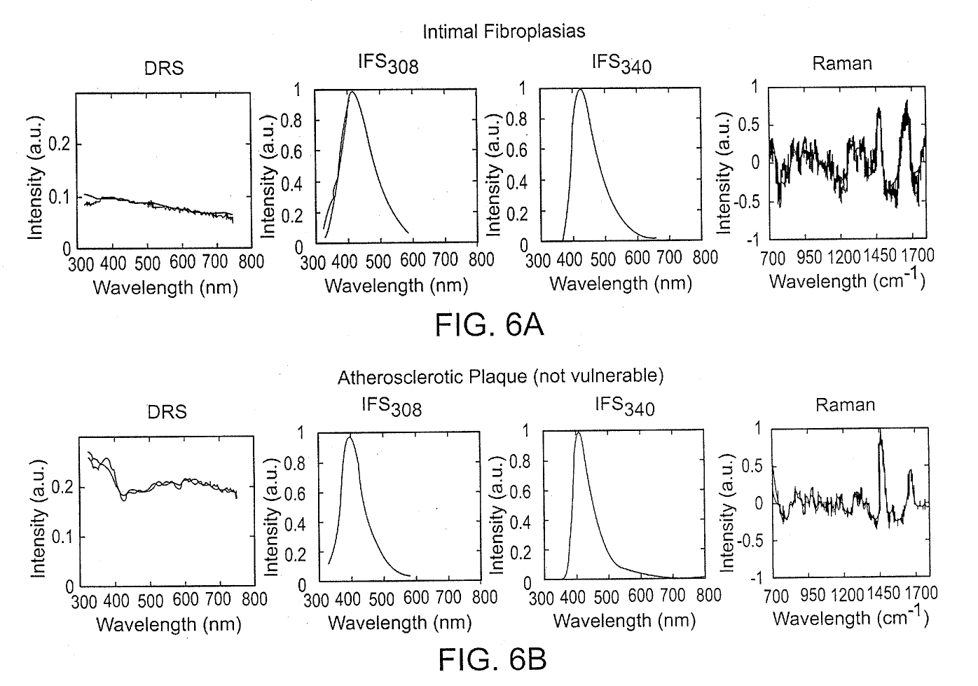

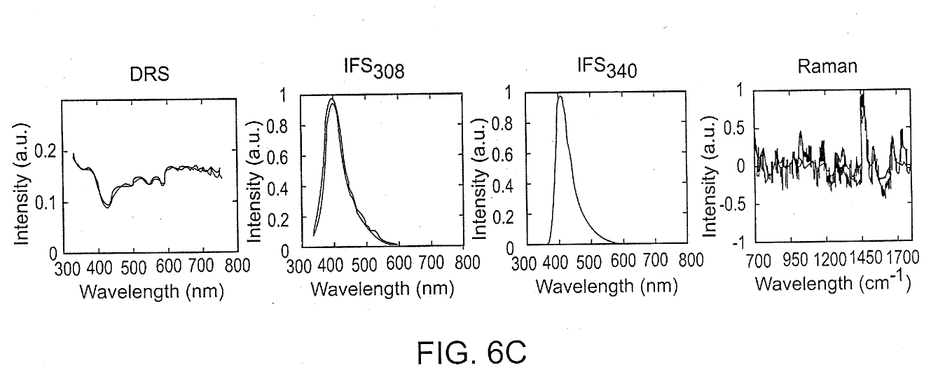

FIG. 6A-6C shows the spectroscopic data and model fits for three different artery lesions, an intimal fibroplasia (A), a non-vulnerable plaque (B) and a vulnerable plaque (C). All of the MMS spectra can be fit very well using the previously described models.

Four spectral parameters were correlated with the histopathologic features of vulnerable plaque: DRS scattering parameter A and hemoglobin concentration; an IFS parameter ρ=C₃₀₈/C₃₄₀, where C₃₀₈ and C₃₄₀ are the contributions of the more blue-shifted MCR basis spectra; and the Raman parameter Σ=CC+FC/NC, where CC and FC/NC are the relative contributions in the Raman spectra of cholesterol crystals and foam cells+necrotic core, respectively. The diagnostic potential as it relates to assessing plaque vulnerability for each of the spectral parameters will be discussed separately in the next paragraphs.

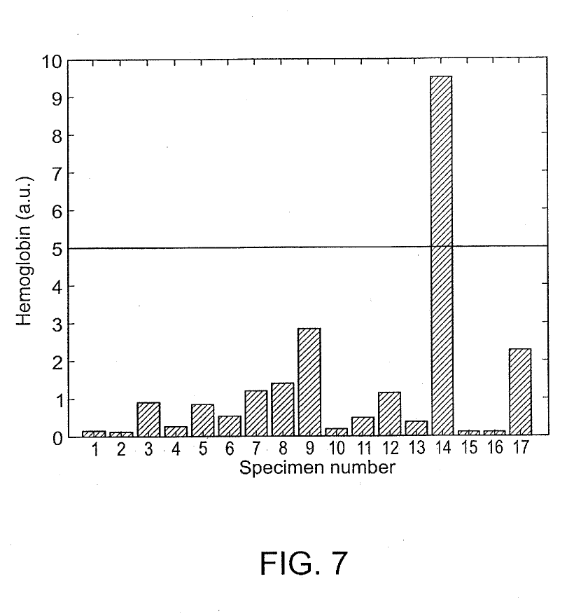

As described earlier, intraplaque hemorrhage is a marker of plaque vulnerability. Histopathology indicates that the lesion in specimen #14 is the site of acute intraplaque hemorrhage (Table 5); whereas the other lesions not hemorrhagic. FIG. 7 displays the hemoglobin concentration fit parameters of the 17 specimens obtained from the DRS spectra. The lesion in specimen #14 exhibits a distinctly high c_{Hb}, and a threshold value of c_{Hb}=5 separates it from the remaining lesions. This suggests that the concentration of hemoglobin inside the arterial wall, measured with DRS to sense acute intraplaque hemorrhage.

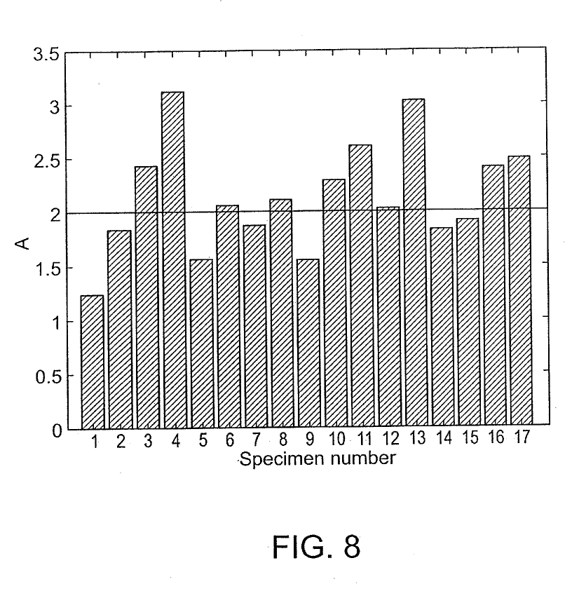

Superficial foam cells are important in assessing plaque vulnerability as they are often present in the fibrous cap near plaque erosions and ruptures, and are a likely source of MMPs that degrade the fibrous cap and lead to plaque rupture. FIG. 8 displays the DRS scattering parameter A (relative units) for the 17 specimens. Foam cells are present in all 10 lesions with A>2, where they occur at an average depth of 250 microns below the intimal surface of the plaque (Table 5). In contrast, foam cells are observed in only 2 of the 7 lesions with A<2, and these foam cells tend to reside deeper in the plaque, at an average depth of 1100 microns (Table 5). Given the several hundred micron penetration depth of DRS at visible wavelengths, DRS does not sense such deep foam cells, which are not clinically relevant to plaque vulnerability. Hence the scattering parameter A appears to be a measure of the presence of superficial foam cells. The correlation of A with foam superficial suggests that the presence of foam cells near the tissue surface can markedly enhance scattering, and that foam cells, which contain a high concentration of lysosomal vesicles, are strong light scatterers. In addition this data suggests that, using parameter A, the method differentiates the presence of foam cells from that of necrotic core, which Raman spectroscopy alone cannot do.

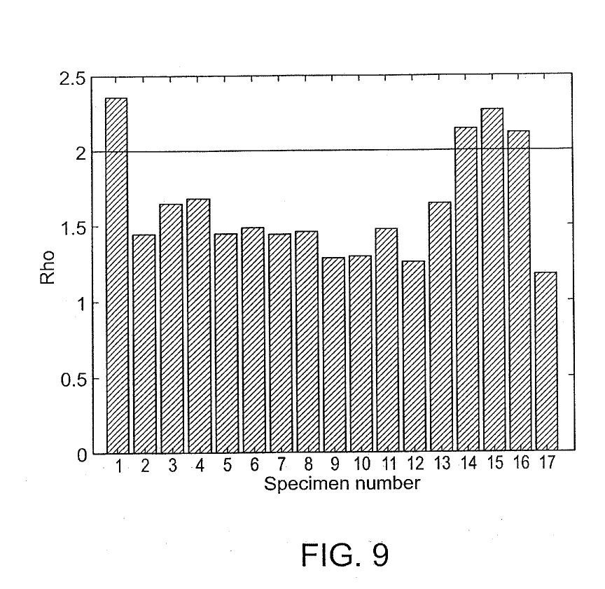

As discussed above, an important feature of vulnerable plaque is the presence of a thin fibrous cap. A cap thickness of less than 65 μm is an established criterion of vulnerability. IFS spectra at 308 and 340 nm excitation wavelengths were obtained to parameterize fibrous cap thickness. Two MCR spectral components to be associated with collagen and/or elastin, structural proteins that characterize the upper layers of both normal artery (normal intima) and atherosclerotic lesions (fibrous cap). Comparing the MCR spectra to the known spectral of those fluorophores, the red-shifted spectrum resembled elastin while the blue-shifted spectrum is similar to collagen (FIG. 5). The corresponding fit coefficients, C₃₄₀ and C₃₀₈, relate to the amount of collagen present within the tissue volume sampled. The sampling depth with 340 nm excitation (˜60 μm) is greater than that with 308 nm excitation (˜50 μm). Thus, C₃₄₀ provides information about collagen and elastin distributed over a much greater depth compared to that provided by C₃₀₈. Hence, the ratio ρ=C₃₀₈/C₃₄₀ can provide information about the thickness of the fibrous cap. FIG. 9 plots ρ for the 17 specimens. Lesions with the highest values (ρ>2, specimens #1 and 14-16) have the lowest average intimal or fibrous cap thicknesses, all below 50 μm. Conversely, for each of the remaining specimens, for which ρ<2, the average cap thickness is greater than 50 μm. The one exception to this is Specimen #17, an ulcerated plaque, which has a variable fibrous cap thickness, ranging from 0 to 200 μm, and yet it has a ρ<2. Nevertheless, these results indicate that a threshold value ρ=2 can be used to identify thin fibrous caps.

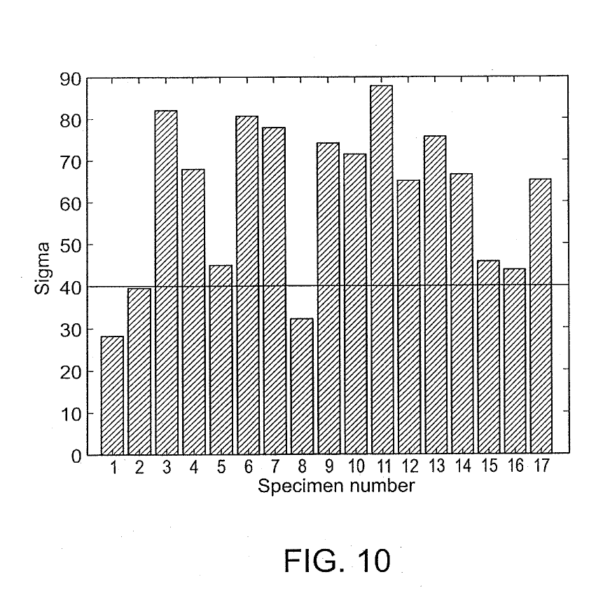

For Raman spectroscopy, the parameter Σ=CC+FC/NC is an indicator of the presence of necrotic material, foam cells and cholesterol crystals. The values of Σ for the 17 carotid artery specimens are plotted in FIG. 10. Specimens rich in foam cells or necrotic core exhibit larger values of Σ. A threshold value of Σ=40 separates specimens of low and high overall lipid content. The only exceptions are specimens #14 and #15, which have high values of Σ although histopathology indicates the absence of foam cells and/or necrotic core. These two specimens are fibrotic-sclerotic plaques. They are morphologically unusual, demonstrating a well-developed fibrous cap but lacking an extracellular necrotic core and cholesterol crystals. These can be viewed as end stage plaques.

The key spectroscopic parameters obtained from IFS, DRS and Raman spectroscopies are displayed together in Table 6 for all 17 specimens. This method uses yes/no results based on the threshold values rather than numerical values. Each column represents a spectroscopic marker of a histologic feature of vulnerable plaque: Hb, indicative of intraplaque hemorrhage; scattering parameter A, indicative of foam cells close to the surface; ρ, indicative of fibrous cap thickness; and Σ, indicative of the build up of necrotic core material. Note that 3 of the 4 vulnerable plaques can be identified by detecting a thin cap (ρ>2) together with another parameter such as A or Σ.

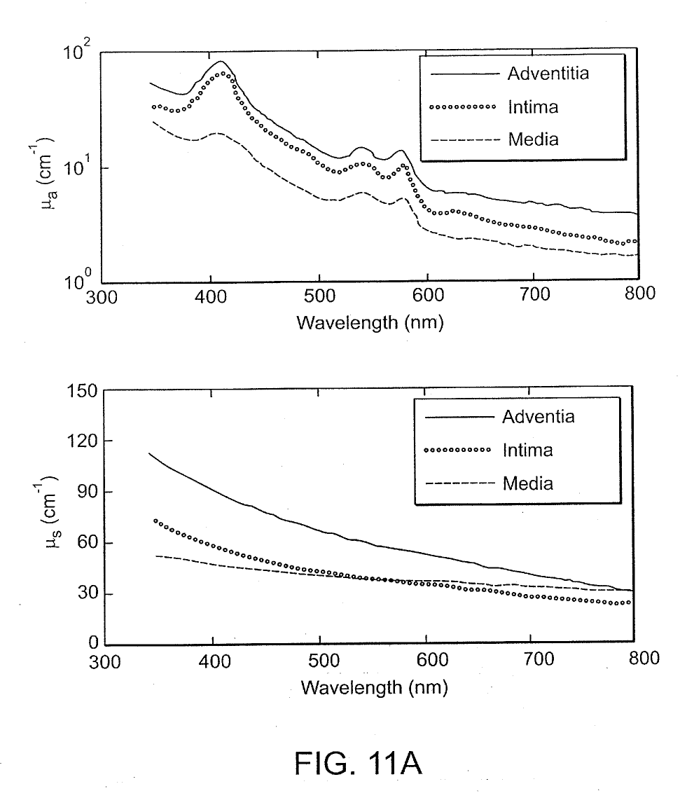

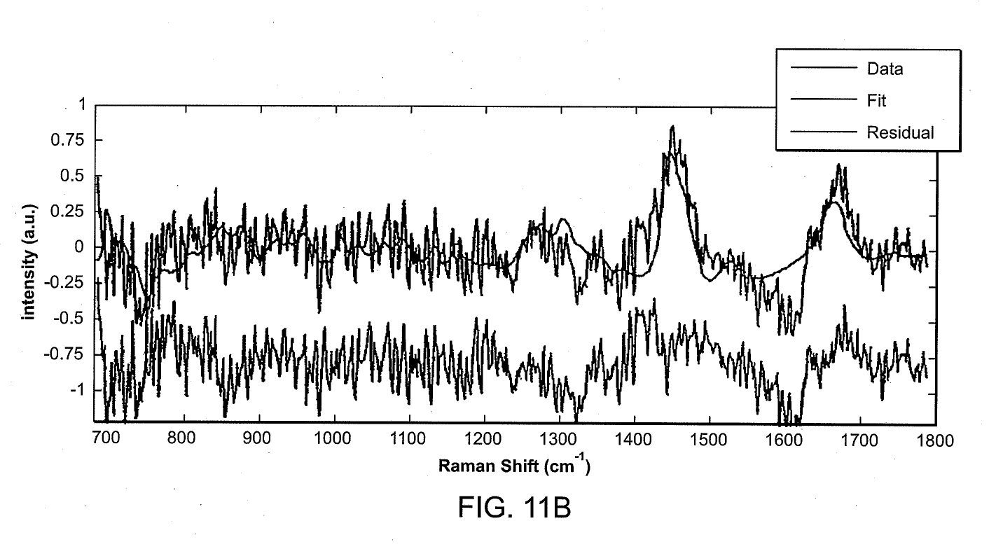

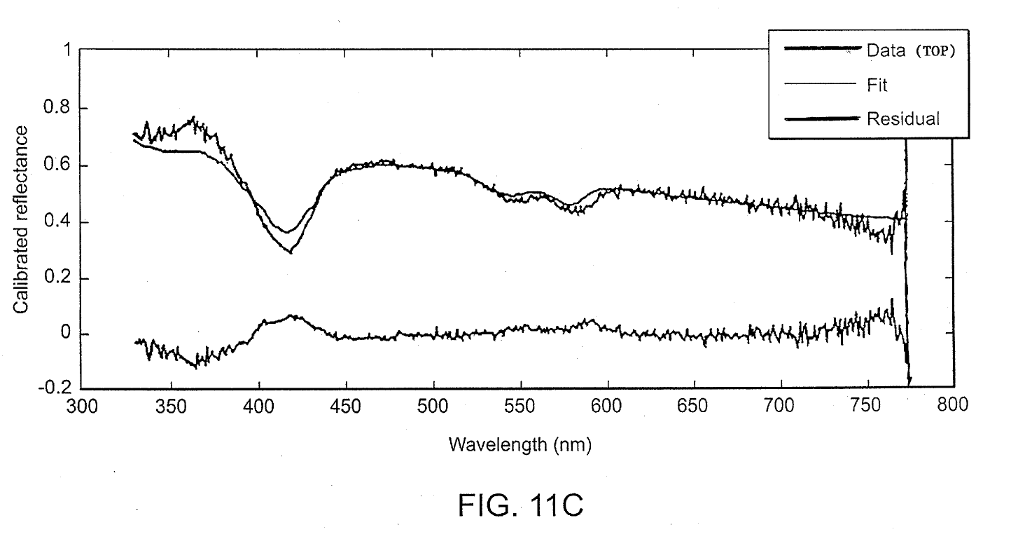

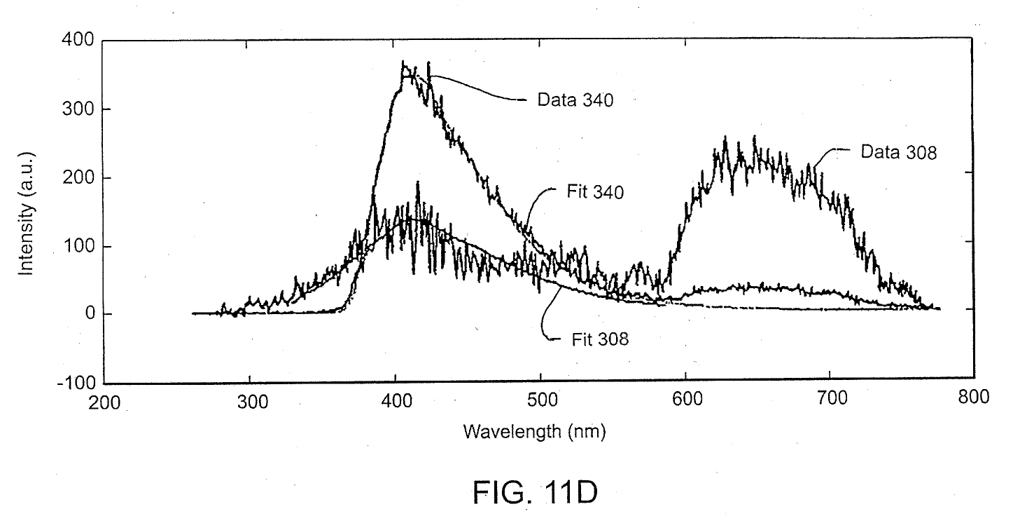

The ability of MMS to provide depth-sensitive information is more relevant to measurements of atherosclerosis than those of breast cancer because of the layered structure of the arterial wall. Define the optical penetration depth as the depth at which the power of light incident on a tissue sample falls to 1/e of its incident value. Generally the optical properties of aorta indicates penetration depths of about 90, 150 and 1200 microns for light of wavelengths 308, 340 and 830 nm, respectively. The penetration depths at different IFS wavelengths were measured by incrementally stacking 20 μm thick sections of aortic media. The FastEEM probe tip was placed in contact with the tissue and the transmitted power was measured as a function of tissue thickness. The penetration depths at 308 and 340 nm were measured as 85 and 105 μm, respectively. These values correspond with prior results especially noting the variability of human tissue. They also agree with estimates obtained from the formula δ=1/μ_{eff}=1/\sqrt{3μₐ(μₐ+μ′ₛ)}, using the known scattering and absorption properties of arterial tissue at different wavelengths; FIG. 11A gives the μₐ and μₛ′ in the visible wavelength range.

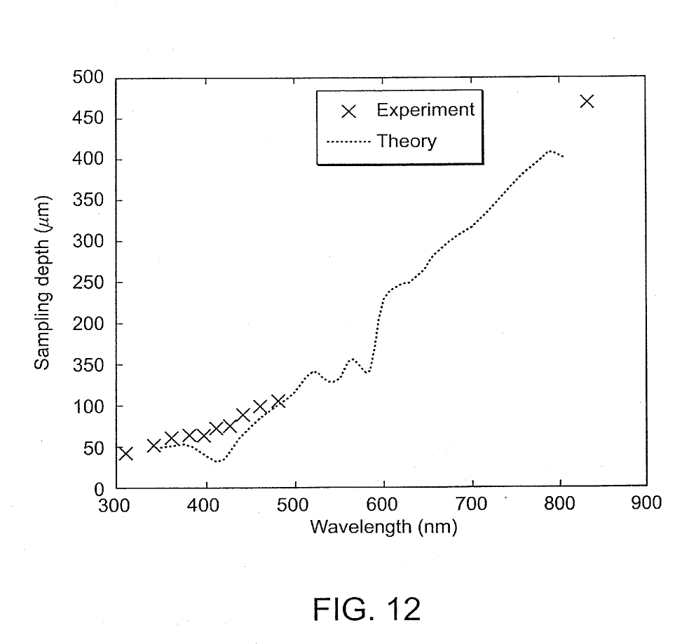

Note that in the single-ended geometry of our artery measurements (i.e. the probe both delivers and collects light at the same position) the sampling depth, can be defined as 1/δₛ=1/δₑₓ+1/δₑₘ, where δₑₓ and δₑₘ are the penetration depths of the excitation and emission light, respectively. The sampling depth characterizes the attenuation of both the excitation and the emitted light, which can be at a longer wavelength, as in the case of fluorescence or Raman scattering. Thus the sampling depths of IFS₃₀₈ and IFS₃₄₀ are much smaller: 50 and 60 μm, respectively, taking into account the longer wavelengths of the emitted light. A previous measurement established a sampling depth of 470 μm for Raman spectroscopy of artery using 830 nm excitation. In the following, use 50, 60 and 470 μm as the sampling depths at 308, 340 and 830 nm, respectively. Note that the definition of penetration as the length where light is attenuated to 1/e of its original value is somewhat arbitrary and that, optionally the device can sample deeper than those values. Similarly, different wavelength regions of the diffuse reflectance spectra sample tissue at different depths. In general, short wavelength IFS (308 nm, in particular) provides information about the top layer (intima/fibrous cap), longer wavelength IFS samples somewhat deeper, and Raman spectroscopy has the greatest sampling depth. FIG. 12 gives the sampling depths at various wavelengths in the range 308-830 nm, comparing values from our experimental studies those calculated from the literature (the emission wavelength is chosen to be the same as the excitation so δₛ=δₑₓ/2).

Multimodal spectroscopy (MMS) is a spectral diagnosis technology that combines spectroscopic results from TMS and Raman spectroscopy to provide more accurate disease diagnosis and a more comprehensive picture of biochemical, morphological and metabolic state of the tissue as it relates to disease pathogenesis and pathophysiology. FIGS. 11B-D illustrate in vivo Raman (FIG. 11B) diffuse reflectance (FIG. 11C) and intrinsic fluorescence (FIG. 11D) spectra taken of a femoral artery. The artifact between 600 and 700 nm in the IFS spectrum is due to the surgical light in the room which can be turned off during use.

The results have demonstrated that combining information from Raman, fluorescence and reflectance spectroscopies provides better diagnostic accuracy than that provided by any one of the spectroscopic techniques independently, and that differences in sampling volumes can be used to advantage for depth sensing.

The present invention relates spectroscopic diagnosis of a wide range of diseases including oral, esophageal, colon and cervical cancer, as well as the diagnosis of vulnerable atherosclerotic plaque and breast cancer. A preferred embodiment spectroscopically extracts biochemical, morphologic and metabolic information related to features of plaque vulnerability or predictive of breast cancer. More than rendering precise disease diagnoses, the system extracts accurate biochemical, morphologic and metabolic information about tissue composition. The system stores IFS morphological basis spectra using microspectroscopy, and performs ex vivo and in vivo tissue measurements using DRS, IFS, and Raman spectroscopic techniques.

Combined MMS spectral data provides insight into depth dependent morphological features of breast cancer (collagen) and vulnerable plaque (fibrous cap thickness and superficiality of foam cells). These measurements simultaneously collect and analyze Raman, DRS and IFS spectra from the same spot without registration errors using an MMS probe.

Quantitative information about biochemical and morphological tissue components are provided from DRS and Raman spectra using basis spectra in our linear combination model. IFS can also provide quantitative information. Meaningful data modeling can be obtained using fluorescence basis spectra measured from biochemical and morphologic tissues structures measured in situ uses the IFS technique to remove the artifacts of tissue absorption and scattering. This can be useful as basis spectra obtained by microspectroscopy of thin (<6 μm) tissue sections or cell cultures can have little or no scattering or absorption effects, and thus may not model uncorrected raw fluorescence spectra as well as IFS spectra.

To build representative libraries of basis spectra, 50-100 spectra were acquired each from a variety of tissue and cellular sources. Tissue handling and preparation methods can lead to spectral distortions. For example, increased absorption has been observed in frozen-thawed tissue, possibly the result of red blood cell lysis, with a concomitant decrease in tissue fluorescence. These changes are less significant in artery wall than in epithelial tissues. Several steps are taken to minimize these artifacts in the collection of IFS basis spectra. First, all IFS basis spectra are collected from freshly excised tissues within 30-60 minutes of excision.

In the case of artery, basis spectra are obtained initially from cryostat sections of fresh tissue that has been immediately snap frozen in liquid nitrogen. Basis spectra are obtained on these sections within minutes of preparation. The passively thawed frozen sections maintained in a humid chamber to prevent drying.

Optionally, basis spectra obtained either from fresh tissue sections (or short term organ cultures) maintained in a balanced electrolyte solution such as Hanks Balanced Salt solution at neutral pH. Under these conditions it is known that tissue remains viable for at least 90 minutes, with minimal changes in fluorescence. Basis spectra can also be obtained from live human cell cultures, where appropriate, to provide a relatively pure population of cells. Cell cultures from which basis spectra may be obtained for artery studies include primary cultures of normal human endothelial and smooth muscle cells and various cell culture models of foam cells, such as LDL fed human alveolar macrophages. Cell cultures from which basis spectra may be obtained for the breast studies include primary cell cultures of normal breast epithelial cells, myoepithelial cells and fibroblasts and human breast cancer cell lines.

The basis spectra can be collected using a confocal microscope adapted for TMS microspectroscopy. A confocal fluorescence system uses excitation light generated by the FastEEM instrument. The excitation light from the FastEEM is delivered from a 200 um fiber, focused to 100 um aperture and collimated. The collimated light is delivered down to the objective using a neutral density beam splitter (90/10) and collected light from the thin tissue is be focused to a confocal pinhole. This light is delivered to the FastEEM spectrograph and CCD via optical fibers. The microscope stage is programmed to FastEEM scan in the features of interest. A bright field image of the specimen is obtained and used for registration between pathology and spectroscopy. The FastEEM software is synchronized for operation between the microscope and FastEEM excitation source and CCD camera.

With the library of biochemical and morphological basis spectra morphological basis spectra (of such structures as foam cells in atherosclerosis and epithelial cell nuclei and cytoplasm in breast cancer) are fit with the same linear combination method used previously for Raman spectroscopy, using biochemical basis spectra to determine their precise chemical composition and identify the fluorophores characteristic of each structure. The basis spectra are also fit to ex vivo IFS tissue spectra, and quantitative information about the presence of fluorophores (tryptophan, collagen, elastin, NADH, FAD, β-carotene) and the morphologic structures they comprise, is extracted. Using this quantitative spectral information obtained from all three spectral modalities, an automated method to characterize morphological components associated with disease state, including their depth profiles, is provided. Quantization of the biochemical and morphologic composition of the tissues is incorporated into algorithms for the diagnosis of vulnerable plaque and breast cancer. Similar basis spectra libraries, spectral models and diagnostic algorithms are used for cancers of the oral cavity, colon, bladder and cervix.

Using at least 200 spectra each from ex vivo fresh arterial (carotid and femoral) and breast tissues from at least 40 different patients spectra are acquired using the MMS instrument using the integrated MMS probe. The location of the spectroscopic site is established by attaching a metal sleeve to the probe that can make a shallow incision around the site. After removing the probe, the location can be marked with an ink dot. The sample can be fixed in formalin and submitted for histopathological examination, by a pathologist. Both spectral analysis and quantitative image analysis (QIA) of the samples is performed in parallel, using the same tissue site for both measurements.

To evaluate the depth sensing capabilities of different fluorescence excitation wavelengths, Monte Carlo models are employed to simulate light propagation within tissue. Monte Carlo models can have simple layered structures with physiological dimensions and optical properties to simulate light propagation in the normal arterial or breast tissue. Optical properties can be measured with an integrating sphere. The spatial distribution of morphological features associated with vulnerable plaque or breast cancer are estimated using QIA software. This information, along with the IFS basis spectra, are used as input into fluorescence Monte Carlo models to evaluate the ability of different excitation wavelengths to probe morphological structures such as foam cells and necrotic core.

DRS provides information about the presence of Hb, indicative of thrombus or intraplaque hemorrhage, and the amplitude of the scattering coefficient A is related to the presence of foam cells and their depth within the artery wall (superficiality). IFS provides information about fibrous cap thickness through the ratio of MCR components at 340 to 308 nm excitation. Raman spectroscopy also provides information related to the presence of foam cells or necrotic core. Thus MMS modalities provide important diagnostic parameters related to collagen (Raman and IFS), diffusive scattering (DRS) and NADH (IFS) that are of use for breast cancer diagnosis.

There are additional correlations between IFS and DRS-measured parameters and key morphological features of breast cancer and vulnerable plaque. For example, detection of 13-carotene by DRS can be a strong marker of tissue necrosis and extracellular lipid pools. Tryptophan is another fluorophore that plays an important diagnostic role in both atherosclerosis and breast cancer.

Fit coefficients from MMS morphological models can be used to predict disease/tissue parameters using logistic regression. These fit coefficients can be used as parameters for an algorithm for distinguishing vulnerable and non-vulnerable plaque and the full spectrum of breast lesions, both benign and malignant.

Spectroscopic instrumentation for MMS can comprise a combined instrument in which a clinical Raman system and a FastEEM are linked together for use with a single combined spectral probe. Alternatively a smaller integrated clinical instrument for a variety of clinical studies involving atherosclerosis, breast cancer Barrett's esophagus and oral cancer. A number of specialized MMS spectral probes can be used for front-view, side-viewing and circumferential imaging modes. See for example U.S. application Ser. No. 10/407,923 filed on Apr. 4, 2003, the entire contents of which is incorporated herein by reference. The measurement for breast cancer and atherosclerosis can be obtained using two independent instruments and separate spectral probes. Due to the differences in these probes, which determines the light collection efficiency, it is preferable to use a single probe. This will eliminate registration uncertainties between Raman and DRS/IFS data and ensure that illumination areas will be the same. This instrument provides the full, range of fluorescence excitation wavelengths and can include a front-looking MMS spectral probe.

A combined instrument can use a FastEEM (See U.S. Pat. No. 6,912,412 incorporated herein by reference) and Raman system combined under a single LabView software program that synchronizes the operation of both units. This instrument collects a set of IFS spectra and a DRS spectrum in 0.2 seconds, followed by collection of a Raman spectrum in 1 second, for example. Excitation light from FastEEM and Raman sources is coupled into a single tapered fiber with 0.22 NA. The tapered fiber has a 600 μm core diameter at one end allowing up to four excitation inputs and can be drawn down to a single 200 μm core for use at the distal end of the probe. For ease of fabrication, MMS probes can be assembled with 15 collection fibers surrounding the central excitation fiber. Alternatively a reduced diameter device has 9 fibers around a single fiber in the probe. The 15 fibers are split at the proximal end so that 10 of the 15 fibers are coupled into the Raman spectrograph while the remaining 5 fibers are coupled to the FastEEM spectrograph. The collection fibers have a core diameter of 200 μm with 0.26 NA. High NA Anhydroguide G fibers can be used in the Raman instrument. They are well suited for near IR wavelengths but have a 40-50% transmission loss in the 300-400 nm region. The Superguide G fibers used in FastEEM have negligible transmission losses in the same UV wavelength range, but low NA. In spite of transmission losses in Anhydroguide G fibers, the spectral quality is not significantly reduced, due to the strength of the fluorescence and reflectance signals at these wavelengths. In one embodiment of an MMS probe, both Superguide and Anhydroguide fibers are used in a single probe to provide a baseline performance level with the optimum transmission properties.

Of the three spectral signals (Raman, DRS and fluorescence), Raman is typically the weakest. Thus, a spectral probe capable of collecting high-quality Raman spectra should easily collect fluorescence and reflectance spectra as well. The spectral probe design for the combined instrument is single-ring front-viewing Raman probe.

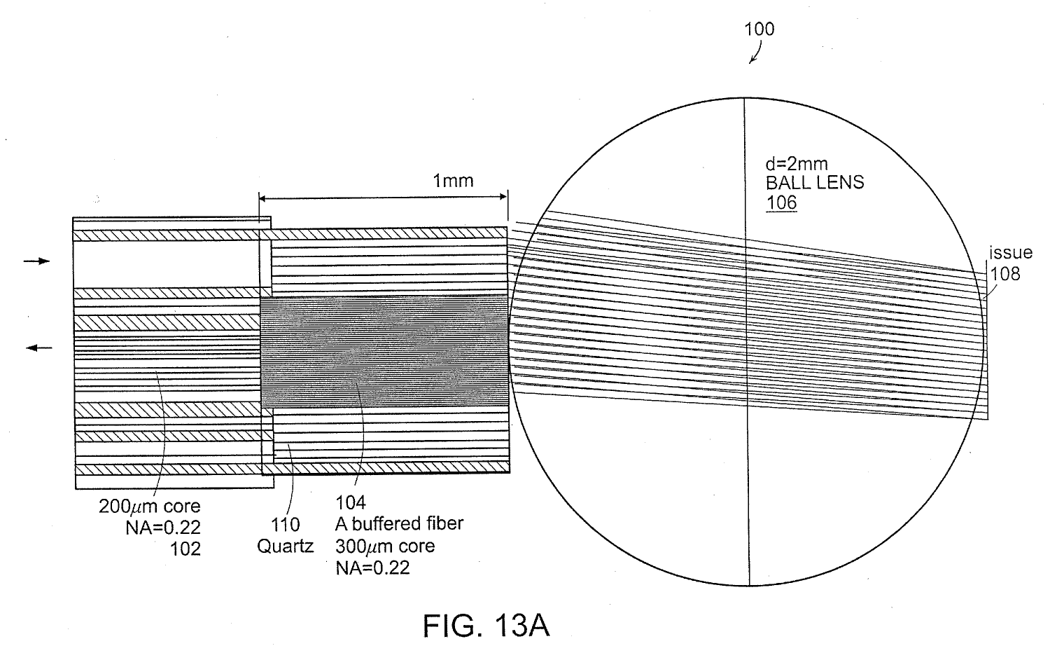

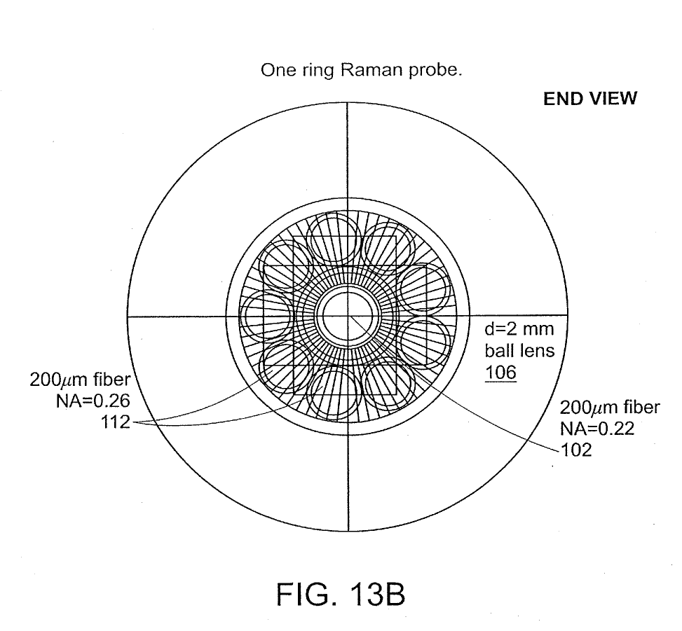

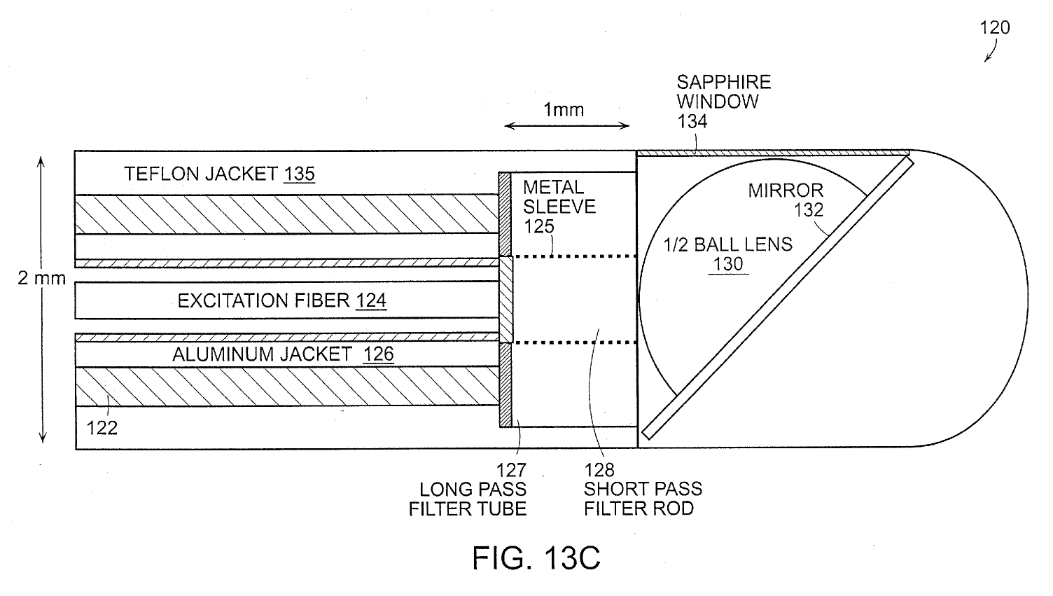

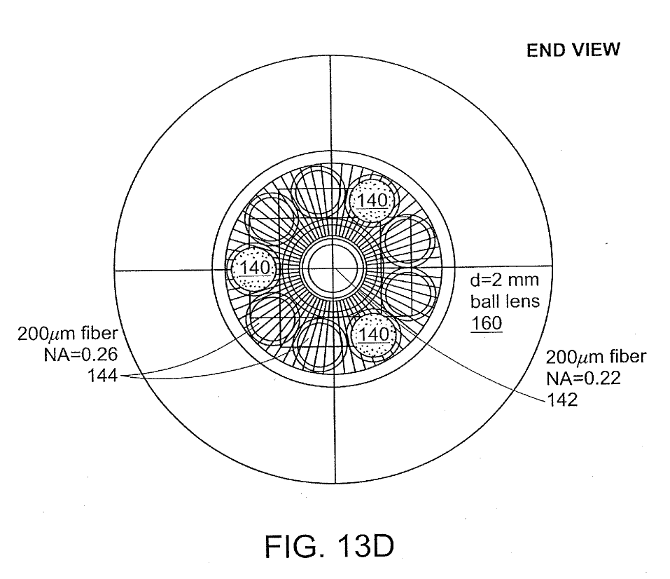

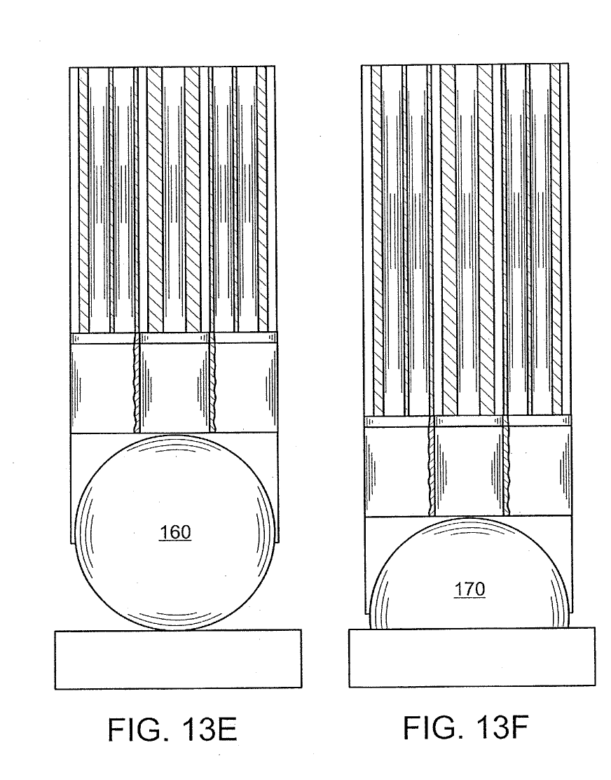

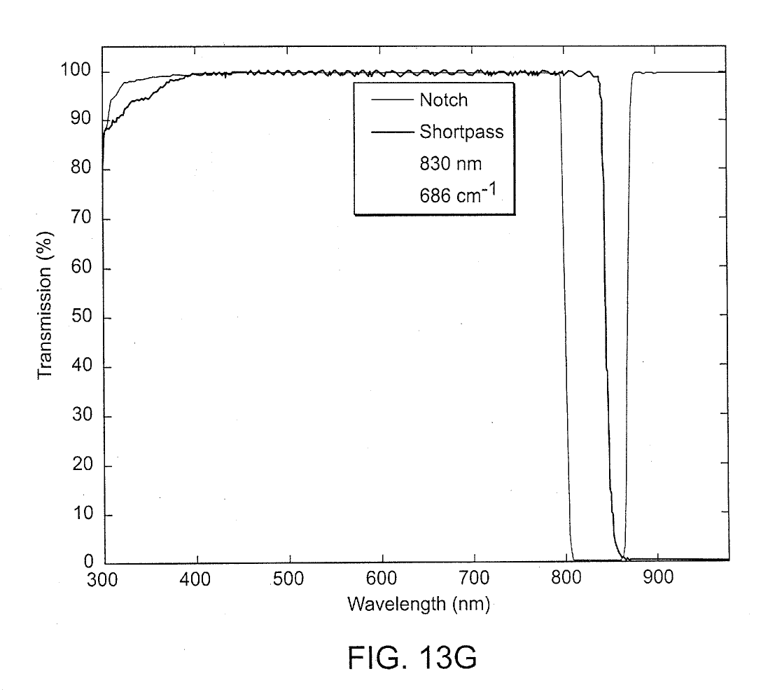

Placement of filters and ball lens, can be the same as the Raman probe, but the filter characteristics has tighter specifications when used with all three spectral modalities. FIG. 13A illustrates the details for a reduced diameter 9-around-1 probe 100 and excitation/collection trajectories through a ball lens 106 that contacts tissue 108. Similar to the Raman probes, the filter module has a filter rod 104 placed on the delivery fiber with transmittance from 300-830 nm and no transmittance (<1%) beyond 850 nm. A filter tube placed on the collection fibers has transmittance from 300-810 nm and from 850-1000 nm and with a narrow 40 nm band centered at 830 nm having low transmittance. An end view of the probe is shown in FIG. 13B with collection fibers 112 positioned in a circular array around central excitation fiber 102. A side looking probe 120 is shown in FIG. 13C in which a half ball lens 130 is in contact with a mirror 132 to reflect light from excitation fiber 124 and filter rod 128 through sapphire window 134. Light returning from the tissue such as artery wall is reflected into collection fibers 122 through long pass filter tube 127. A metal sleeve 125 surrounds filter 128. An aluminum jacket surrounds the excitation fiber 126. A Teflon jacket 135 provides the cylindrical tube that forms the outer wall of the catheter.

In FIG. 13D an end view of a design in which a first group of 3 collection fibers 140 are used to collect reflected light and 3 pairs of fibers 144 collect the Raman light passing through ball lens 160. The central fiber 142 directs light through the forward looking probe with lens 160 in FIG. 13E or half ball lens 170 of FIG. 13F. The filter system used in the probe is shown in FIG. 13G.

The wavelength-dependent sampling volume and depth of penetration of the probe can be determined with tissue phantoms and/or thin sections of tissue. The diameter of the excitation spot illuminating the tissue can be approximately equal for all wavelengths; however, the tissue penetration depth is different for different excitation wavelengths. Because the spot diameter and penetration depth are important for diagnostic algorithms and they are measured and checked with Zemax optical design models and Monte Carlo models.

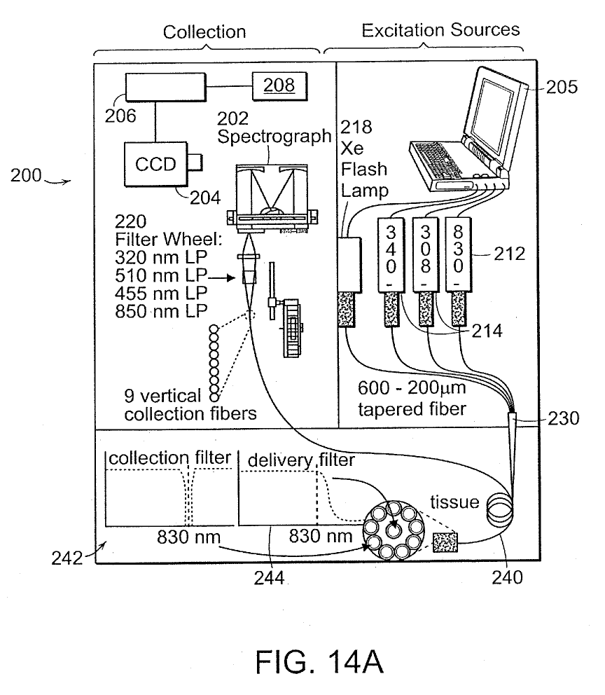

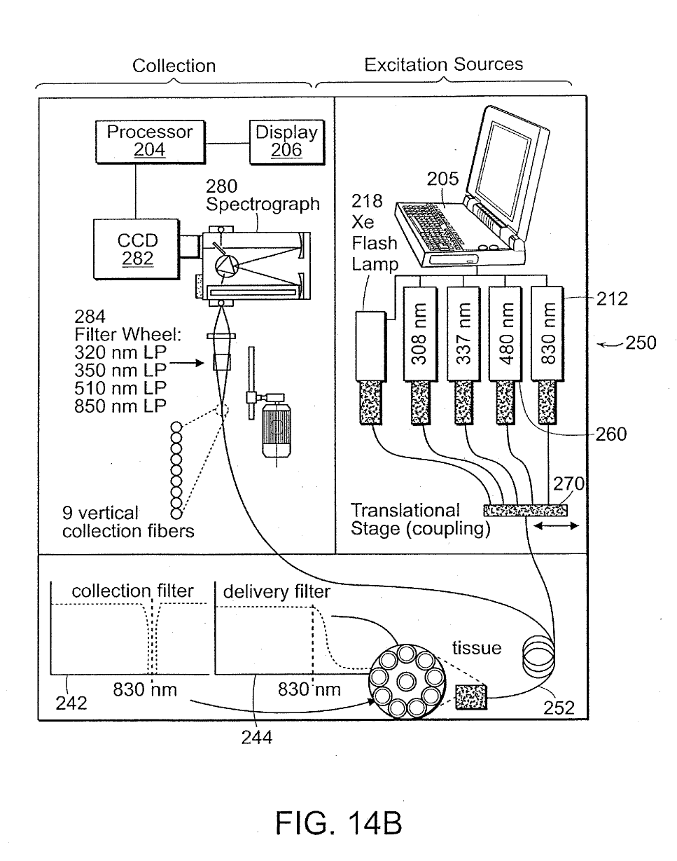

A compact portable MMS instrument that incorporates all three spectroscopic modalities (DRS, IFS and Raman) is shown in FIG. 14A. The fourth modality, LSS, requires no extra instrumentation. A preferred MMS instrument 200 uses solid state light emitting diodes, reducing the instrument size, complexity and cost, and eliminate many maintenance issues related to excimer laser and dye cell operation. The MMS instrument can employ a common spectrograph 202 and CCD 204 for all spectral acquisition.

To accommodate the requirements for using all three spectroscopic modalities, spectra are collected over the wavelength range 300-1000 nm. Excitation light for each modality is delivered sequentially to the sample, and fluorescence, DRS and Raman spectra are acquired. This is followed by real-time analysis of the data, during which IFS spectra are derived from the fluorescence and DRS spectra. The information from the different modalities provides depth-sensitive, complementary chemical and morphological information on tissue sites.

The measurements include IFS spectra excited at 308 and 340 nm, DRS and Raman spectra. The combined TMS/Raman instrument is used for FastEEM fluorescence excitation wavelengths to determine the diagnostic value of the various excitation wavelengths. The most appropriate two or three fluorescence wavelengths can be used in the integrated system.

Data acquisition, analysis and tissue characterization preferably occurs in 5 sec or less. Triggering of the light sources is accomplished by means of a National Instruments Timer/Counter card and a Princeton Instruments CCD controller, respectively. The sequence of operation can be controlled by computer 205 as follows: (1) Initialize CCD for spectral acquisition; (2) open shutter for the CCD and activate insertion of appropriate collection filter; (3) trigger light source (LED, diode laser or flashlamp); (4) acquire spectrum; (5) close shutter; (6) read/transfer data and store in computer 206 and display at 208. The time for acquiring all spectra depends upon the excitation power, thus the exposure time can be adjusted to accommodate signal levels.

Separate excitation and reflectance sources can be used for each spectroscopic modality. Laser emitting diodes 214 (˜1 mW) provide fluorescence excitation light at 308 and 340 nm, a 60 W xenon flashlamp generates a continuous spectrum from 300-1000 nm for DRS, and a laser diode 212 at 830 nm (500 mW) will generate the Raman excitation light. A flashlamp 218 can be used in the FastEEM, and the 830 nm laser diode in the Raman system. Each of these four light sources can be focused onto separate 200 μm core diameter optical fibers, and then coupled together into a 600-to-200 μm tapered optical fiber The output can be connected to the combined spectral probe via an SMA connector. The system enables fluorescence excitation wavelengths to be added and/or changed.

UV diode sources can be used compact light sources in the 300-340 nm range available. UV light emitting diodes at wavelengths as short as 275 nm or UV LEDs in the 305-360 nm wavelength range can be used. Present 308 nm LEDs produce 1-2 mW of CW power in a bandwidth of 10-15 nm, emitted from a 0.1 mm aperture over a 30° angular range. Because of this large bandwidth, a filter can be used to restrict the light to a 2 nm bandwidth. Thus, under present conditions, ˜1 μJ of 308 nm light can be delivered via 200 micron core, 0.26 NA, fused silica optical fiber in 10 ms, resulting in the acquisition of high SNR fluorescence spectra. Characteristics of 340 nm LEDs are even more favorable.

Each of the spectral probe collection fibers, typically nine, (fifteen in one design) are coupled to an SMA connector mounted on the front panel of the instrument. Long (wavelength) pass filters 220 mounted on a programmable wheel driven by a stepper motor are positioned in the return beam path to prevent Raman and fluorescence excitation light scattered from the tissue from entering the spectrograph. Since the reflectance measurements cover a broad range (300-1000 nm), the acquired spectra contain second order contributions. Taking two reflectance measurements, one with no filter and another with a long pass 500 nm cutoff filter (mounted on the wheel), eliminates these contributions. The unfiltered reflectance provides spectral information below 600 nm, and the filtered reflectance provides information above 500 nm. The Princeton Instruments Spec10:400 BR CCD camera of the Raman system can be coupled to an Acton Research Spectra Pro 150 spectrograph with a grating blazed at 500 nm and 200 grooves/mm. Alternatively two separate gratings or dispersive elements can deliver different light modalities onto separate regions of the detector.

This combination of fluorescence, reflectance and Raman capabilities in one instrument provides a compact clinical instrument. With a single spectrograph/CCD combination, a spectral range of 300-1000 nm is covered, compared to 155 nm in our existing Raman system. This causes an increase in spectral dispersion by a factor of 4.5, and a reduction in system resolution from 10 to 45 cm⁻¹. However, if the spectral resolution degrades the accuracy of the Raman fit coefficients significantly such that diagnostic accuracy is also degraded. A two spectrograph/CCD system can also be used with one spectrograph/CCD combination is dedicated to Raman while the other to fluorescence/reflectance. A high-speed mirror will direct the collected light to appropriate spectrograph/CCD combination.

A further embodiment of a system 250 is shown in FIG. 14B in which a translational stage 270 is used to couple light from the source sequentially into the probe 252. This contrasts with the prior embodiment where the sources are coupled to probe 240 with combiner 230 to provide simultaneous illumination. The delivery 244 and collection 242 filters are shown schematically. Another source 260 is also used and accounted for in the filter which 284, spectrograph 280 and detector 282 system.

The detection of vulnerable plaques, margin assessment in breast cancer and transdermal needle biopsies can be performed using front-viewing, side-viewing or circumferential imaging probes.

Using the integrated MMS system, spectra are collected from several of these margins prior to excision and thus only tissue that would normally be excised during the procedure will be removed. During each procedure, the distal end of the sterilized MMS front-viewing probe is placed in gentle contact with the marginal breast tissue in the surgical cavity under direct visualization while spectra are acquired. All room and surgical lights will be turned off during the measurements. The spectrally examined marginal tissue will then excised by the surgeon and submitted for conventional pathological examination.

Under local anesthesia following a manual incision of the skin, a cannula having a diameter 0.5 to 2 cm is advanced toward the suspect lesion guided under ultrasound or stereotactic mammography. The central channel of the needle contains a circular blade that is used to cut the biopsy and will provide access for the MMS probe. Once positioned in the lesion, a MMS side-viewing probe is inserted in the central channel and acquire a series of spectra as the probe is withdrawn along the opening. The probe is withdrawn and cutting blade replaced and a biopsy is acquired. Biopsies are performed over a 360 degree around the axis of the needle without it being withdrawn with typically twelve cores of tissue are removed using 11 to 14 gauge needles. The excised biopsy specimens are submitted for specimen radiography to document the presence of calcification and then conventional pathology.

A digital photograph of the lesion and probe placement is recorded. Precise registration between the probe location and biopsy site is ensured by immediately scoring a circular region of tissue slightly larger than diameter of the probe with a 1.5 mm punch biopsy. A larger punch biopsy (˜3.5 mm) is used to remove a larger tissue specimen for histopathology and slide preparation. The smaller mark is located later when viewing the slide under the microscope.

A portable tissue scanner was constructed that can scan large tissue specimens (up to 20 cm×20 cm or larger, for example) at high resolution (at least 0.25 mm) in a clinically acceptable time frame (less than 20 minutes). FIG. 15A shows a schematic diagram of a tissue scanner system 400 in accordance with the invention. The scanner employs a unitary multimodal optical fiber probe 410, or probes, that can be employed in a multimodal clinical spectroscopy system for point spectroscopy measurements. Two (or three) optical fiber probes can also be used, one for DRS, another for IFS (and optionally, a third for Raman measurements), at a fixed separation of 0.75 cm to minimize cross talk between the probes. For the single probe system, a probe shown in FIG. 15B using a single light delivery fiber 142, and pairs of separate collection fibers 140, 144 and 146 to collect DRS, IFS and Raman spectra, respectively. This can use a distal filter and lens system as described previously herein with the distal filters configured for each collection frequency range of the different modes.

Each probe can include a fiber bundle with a single central fiber that delivers excitation light to the tissue, surrounded by a ring with a plurality of optical fibers that collect reflected and fluorescent light returning from the sample and transmit it to the spectrograph (all fibers have 200 μm core and NA=0.22), terminated with a transparent, protective optical shield. A 75 W Xenon arc lamp 403 (Oriel Instrument, USA) and power module 404 can be used to generate excitation light for DRS and a 7 mW Q-switched solid state laser 405 at 355 nm (SNV-40E-000, Teem Photonics) with driver circuit 406 is used to generate excitation light for IFS. Other excitation wavelengths can be used for other diagnostic applications. An infrared Raman source 412 can be used for Raman spectral measurements. Signals are collected with miniature spectrometers (USB2000+, Ocean Optics). The spectrometers 407A, 407B, 407C have spectral resolution of 2 nm at full width half maximum (FWHM). The collection fibers 411A, 411B, and 411C are coupled to the corresponding reflectance, fluorescence and Raman spectrometers. The wide area imaging capability is achieved by mechanically scanning optical probes with a long traveling range, XY translation stage 401 and step motors 402 (Applied Motion Products, micro stepper motor: 17-075 and driver: 3540i) in an inverted geometry through a standard glass plate 409 (10×12× 1/16 inches) on which the specimen rests. There is no interference from glass fluorescence with the biomolecular fluorphores of interest, such as collagen and NADH.

The glass plate flattens the tissue surface and provides a reasonably uniform probe-tissue imaging distance. This provides for quantitative measurements, by preserving the key optical characteristics of the probe 410 (spot size and NA), and take full advantage of probe-based spectroscopic models. Excitation beam spot size at the surface of a tissue sample sitting on the glass plate is estimated to be <1 mm. Labview 8.6 (National Instrument, TX) manages the raster scan by commanding the XY stage 401 through PC serial ports and the spectral data acquisitions. Total scanning time for the tissue sample depends on the choice of scan parameters such as excitation power, integrating time, spatial resolution, field of view, etc., which can be adjusted according to tissue type and clinical need. Note that total scanning time includes timing response to start/stop and reverse the stepper motors. The portable device measures 2×1×1 ft, weighs 30 lbs or less and can easily fit in most clinical spaces including patient examination rooms, procedure rooms and operating rooms.

DRS, fluorescence spectra (350-700 nm), and Raman data can be obtained for each spot scanned. After background subtraction and normalization with 20% spectralon white reflectance standards (Labsphere, NH), DRS spectra are analyzed using a mathematical model based on the diffusion approximation of light propagation in tissue. IFS spectra are then obtained, by correcting the raw fluorescence spectra for the effects of tissue absorption and scattering using the corresponding DRS spectra, and analyzed with data processor 408 using a linear combination model based on multivariate curve resolution (MCR), a standard chemometric method. Spectral modeling provides physically meaningful fitting parameters that are quantitative measures the contributions of specific tissue components. These spectral parameters are the basis of decision algorithms used in the diagnosis of breast and other cancers. DRS modeling yields 3 scattering parameters: A, which is related to the amount of Mie scatterers; B, which is related to the size of the scatterers; and C, which is related to the amount of Rayleigh scatterers; and absorption fitting parameters for hemoglobin (Hb) and β-carotene, two well-characterized absorbers in breast tissue. IFS modeling yields fluorescence fitting parameters related to NADH, a cellular metabolite, and collagen, a fluorophore that is more abundant in the fibrous stroma of breast cancer than in normal breast tissue.

DRS and IFS data cubes, i.e. three dimensional arrays of image XY coordinates and wavelength, are obtained during each scan. The overlap regions are then co-registered, without the need for complex mathematical transformation except simple shifting of the XY register in acquired pixels, which was previously obtained from the relative position of the probe during calibration. By registration of DRS and IFS (and optionally a Raman probe) probe positions, the processing and display system 408 can reassemble 2D quantitative hyperspectral DRS and IFS (and Raman) intensity maps of the scanned tissue surface. The spectra are then modeled and spectral fitting parameters extracted on a pixel-by-pixel basis to create quantitative parameter maps. A tissue diagnosis can also be rendered using a parameter-based decision algorithm to create diagnostic maps. Currently data is processed off line, and can take up to a second per data point for model fitting. Real-time (on-the-fly) data processing can be performed by incorporating machine learning algorithms that have been used for spectroscopic modalities. Using a multichannel light collection system as described herein can provide scans of 10 cm² area sections in less than 5 minutes.

Tissue-simulating liquid phantoms were prepared from various mixtures of intralipid (Invitrogen), hemoglobin (Hb) (Sigma Aldrich) or blood, and furan (Sigma Aldrich) to validate quantitative extraction of tissue absorption and florescence properties from the spectral data obtained with the scanner. Self-adhesive O-rings with an internal diameter of 1 cm were arranged on the glass plate to hold droplets of the liquid phantoms in place. Using a microliter pipette, 200 μL of each liquid phantom was carefully placed into the O-rings, forming droplets ˜2.5 mm in depth. Spectralon standards (10% and 20%) were also placed in the field of view, and were used to normalize the spectral data. Spectra obtained from each spot within each phantom were averaged.

Animal tissue measurements were performed to demonstrate high resolution wide-field hyperspectral imaging capability and spectral contrast to distinguish tissue structures with varying optical properties. A clean-cut cross section of an unfixed, frozen-thawed porcine lower leg tissue specimen (˜10 cm in diameter) was used. Before placing the tissue section on the glass plate for scanning, it was moistened with normal saline. Porcine lower leg tissue was used for this study as it has more anatomic detail requiring high resolution imaging and a wider range of tissue types with more varied optical properties than breast tissue.

Human tissue measurements were performed using tissue samples of patients. A paired set of unfixed, frozen breast tissues, one grossly cancerous and another matched grossly normal breast tissue from the same patient, were obtained for scanning on the tissue scanner. To prevent dehydration, the tissues were moistened with a small amount of normal saline. The tissues were placed side-by-side on the scanner glass plate. After scanning, the tissue surfaces scanned were marked with colored colloidal inks to preserve orientation, fixed in 10% neutral buffered formalin, processed and embedded in paraffin, and hematoxylin and eosin stained tissue sections prepared for microscopic examination by an experienced breast pathologist, for comparison with the spectroscopic imaging results.

Measurements can also be performed on excised melanomas in the skin as well as other solid tumor tissues (e.g. lung, liver, kidney). As these methods can sample from tens to hundreds of microns into the tissue, the system provides improved margin assessment in many applications.

A number of measurements were performed to verify system performance. A resolution target was used to demonstrate the imaging capability and to verify spatial resolution of the system. A series of tissue-simulating liquid phantoms was used to validate quantitative extraction of tissue properties from the reflectance and fluorescence spectral data. Moreover, biological tissue was used to illustrate the hyperspectral imaging capability of the system and demonstrate spectral contrast to distinguish tissues with different scattering and absorption properties. Finally, normal and cancer tissues from a breast cancer patient were used to demonstrate the usefulness of the tissue scanner as a cancer margin assessment device.

In the resolution target measurement, a positive multi-frequency grid distortion target (NT46-250, Edmond Optics, FIG. 16A) was placed face-down on the glass plate and a DRS scan obtained from an area larger than 2.5 cm×2.5 cm with excitation power 20 mW, integrating time 20 msec, spatial or image pixel resolution 0.25 mm. Total acquisition time for the DRS image was less than 10 min. At 0.5 μm wavelength, the smallest dot features printed in the target, 250 microns in diameter, can be resolved as shown in the randomly selected DRS image at 510 nm in FIG. 16B. Similar resolution was also found for IFS scanning. The measurements show that the tissue scanner can image a large field of view with sub-millimeter resolution. Preferred embodiments utilize a scanning area of at least 16 cm² in area (4 cm by 4 cm), for example.

Next, a clean-cut cross section of a frozen-thawed porcine lower leg tissue specimen (˜10 cm in diameter) was used to demonstrate scanning of large fields of view and spectral contrast to distinguish tissue structures with different optical properties (FIG. 16C). After placement on the glass plate (FIG. 16D), DRS scans were performed with excitation power 250 mW, integrating time 50 msec and spatial resolution 0.5 mm. Total acquisition time for the DRS image was 30 min. FIGS. 16E-16H show DRS spectral intensity maps acquired with the scanner at selected wavelengths. The DRS spectral intensity map at 487 nm was optimal for distinguishing tissue types with widely differing optical properties in this tissue, including bone, skeletal muscle, fat and connective tissues. The biological tissue measurements show that the tissue scanner can image a large field of view with both high spatial resolution and spectral contrast to distinguish tissues with differing optical properties.

Fifteen liquid tissue simulating phantoms shown in FIG. 17A, composed of a 2% intralipid solution and various amounts of Hb powder to simulate breast tissue scattering and absorption, were prepared and used for DRS calibration. DRS scans with the scanner (FIG. 17B) were performed with excitation power 150 mW, integrating time 100 msec and spatial resolution 0.5 mm. Hb concentration is highest in the first phantom (#1) and last phantom (#15), and progressively decreases from phantom 1 to phantom 14. A 2D DRS scanned image of the phantoms and spectralon standards at a randomly selected wavelength (490 nm) is shown in FIG. 17C. As expected, the reflectance signal intensity was inversely proportional to Hb concentration (due to Hb absorption). That is, the reflectance signal is the lowest in phantoms #1 and #15, and increases with decreasing Hb concentration from phantom #1 to phantom #14. FIG. 18A shows a DRS spectrum of a phantom with 1.8 mg/ml Hb along with its corresponding model fit and residual. FIG. 18B shows Hb concentration computed from the average phantom DRS spectra, which show excellent agreement with the actual phantom Hb concentrations (error≦5%). All calculated scattering-related parameters were constant in all samples. Additional measurements with constant Hb concentration and varying intralipid scatterer concentration showed the computed scattering parameters were proportional to intralipid concentration. The results of these liquid phantom measurements confirm that the tissue scanner can accurately measure a physiologically relevant range of DRS absorption and scattering parameters across a large scanning field of view.

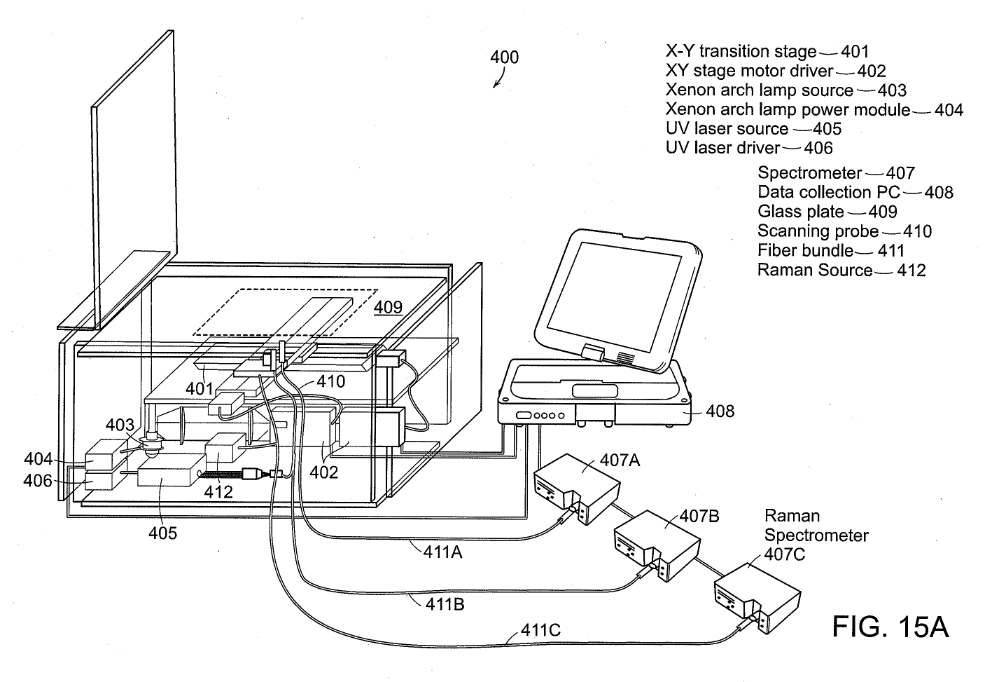

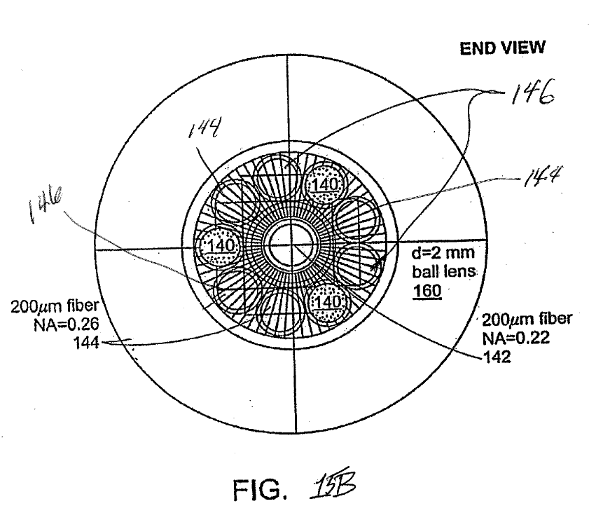

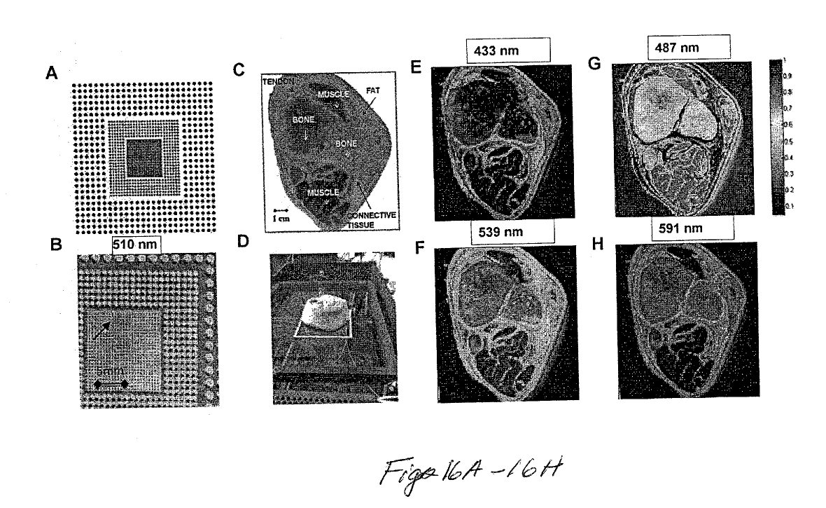

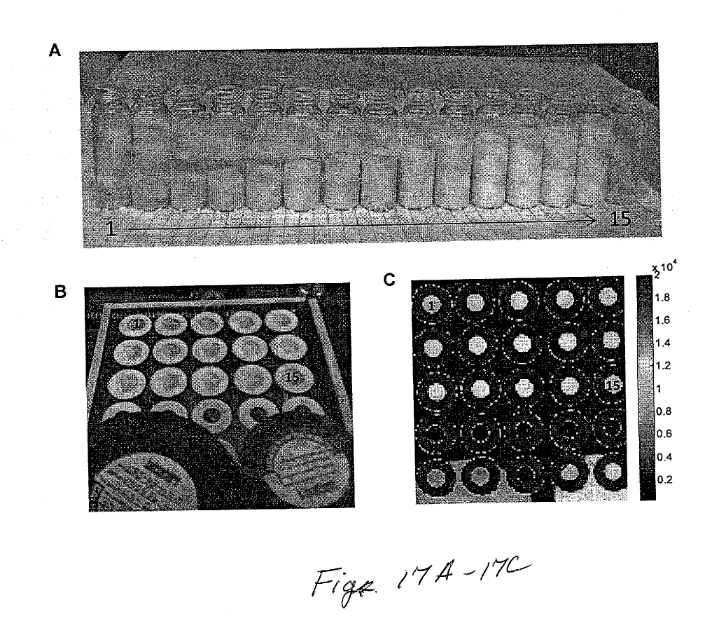

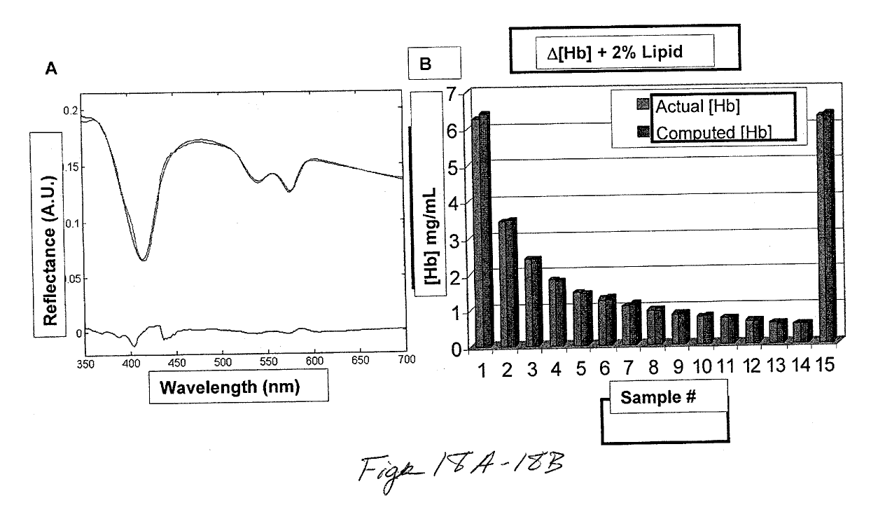

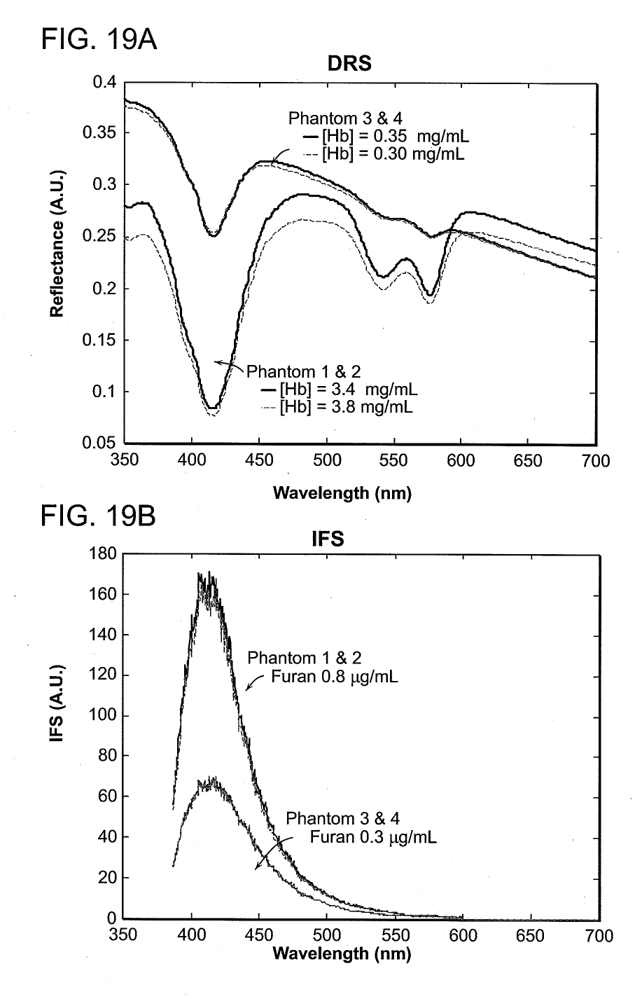

For IFS validation, 4 liquid tissue simulating phantoms were used, consisting of two replicates each of a 1% intralipid solution with two different concentrations of human blood and furan (0.3 and 0.8 μg/mL), which has strong fluorescence in the 400-450 nm region (Table 6). Note that a pin-prick blood sample was used instead of Hb powder to provide additional biochemical fluorophores and scatterers with which to measure DRS correction of the fluorescence spectra for the effects of scattering and absorption. IFS scans were performed with excitation power of 1.5 mW and integrating time of 10 msec. Hb concentrations in the two sets of replicate phantoms were determined from the DRS data and found to be 3.6±0.2 and 0.325±0.25 mg/mL. FIG. 19A shows that the DRS spectra of the 4 phantoms vary primarily with the Hb concentration, whereas the IFS spectra shown in FIG. 19B are largely independent of Hb concentration and depend on the concentration of furan. The results of these liquid phantom measurements confirm that the tissue scanner can accurately measure fluorophores in a variable background of absorption and scattering across a large scanning field of view.

TABLE 6
IFS tissue simulating phantom mixtures of furan, intralipid and blood.

| | Furan | | Hb |
|---|---|---|---|
| | (μg/mL) | Intralipid | (mg/mL) |
| Phantom 1 | 0.8 | 1% | 3.80 |
| Phantom 2 | 0.8 | 1% | 3.40 |
| Phantom 3 | 0.3 | 1% | 0.35 |
| Phantom 4 | 0.3 | 1% | 0.30 |

To demonstrate the capability of the scanner to identify human breast cancer tissue, a paired set of frozen-thawed breast tissues, one grossly cancerous and another matched grossly normal breast tissue from the same patient, were placed side-by-side on the scanner glass plate as close together as possible to minimize the gap between the tissues (FIGS. 20A-20B). DRS and IFS scans were performed by simultaneously scanning the 3 cm×3 cm (at least 9 cm² in area) region of interest, using separate DRS and IFS probes with a probe separation of 0.75 cm and spatial resolution of 0.25 mm per pixel. Excitation power and integrating time were 400 mW and 10 msec for DRS, and 1.5 mW and 10 msec for IFS scanning. Total image acquisition time for 150×150 pixels was 18 mins. 2D quantitative DRS and IFS spectral intensity maps were created. The DRS and IFS spectra were then modeled and fitting parameters extracted to form 2D quantitative parameter maps for comparison with pathology.

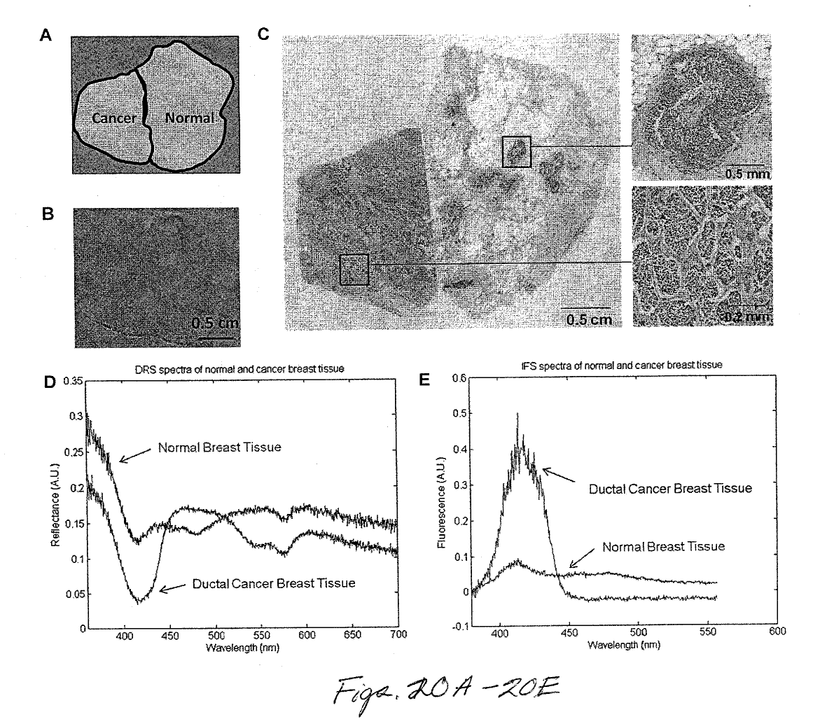

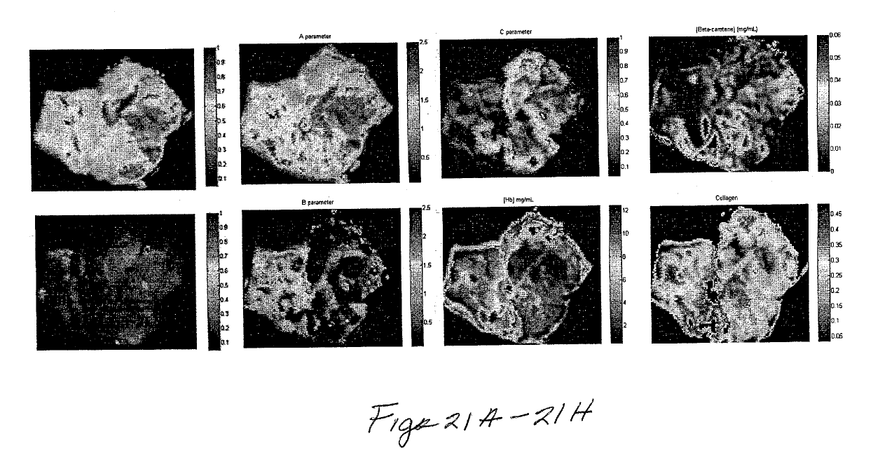

Results of pathology examination (FIG. 20C) confirmed that the grossly cancerous tissue consists largely of breast cancer (invasive ductal carcinoma). Interestingly, the grossly normal breast tissue, although seen microscopically to consist largely of normal breast tissue, contained multiple foci of breast cancer (ductal carcinoma in situ) <1 to 3 mm in diameter. DRS and IFS spectra obtained from scanner image pixels occupied by breast cancer and normal breast tissue (FIGS. 20D-20E) are similar to those we obtained using similar optical fiber probes in a non-imaging multimodal spectroscopy system. However, although individual 2D DRS and IFS spectral intensity maps demonstrate spectral contrast and have the requisite chemical information embedded in them, they do not by themselves reliably distinguish normal breast tissue from breast cancer (FIGS. 21A-21B).

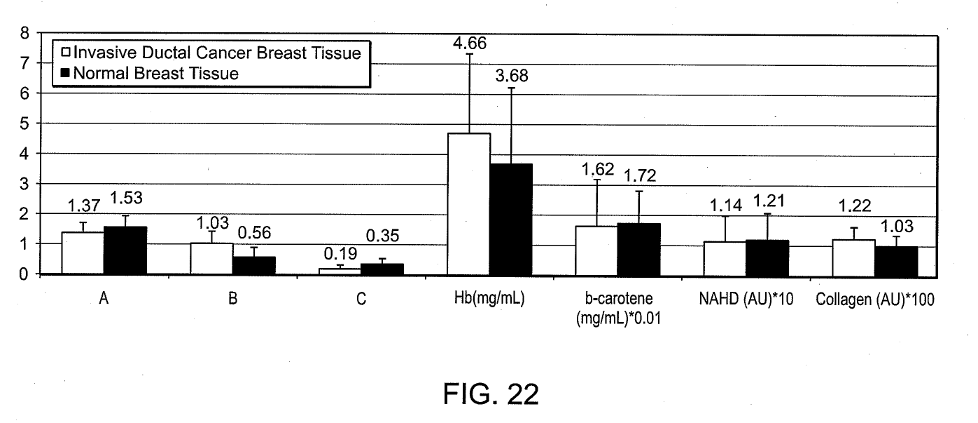

To explicitly distinguish between cancerous and normal breast tissue, employed the physico-chemical fitting parameters extracted from the DRS and IFS spectra. A total of seven parameters were extracted from the spectra and used to characterize the tissue: four DRS parameters (A, B, C, Hb and β-carotene) and two IFS parameters (collagen and NADH). FIG. 22 is a graphical representation of the mean and standard deviation of these spectral parameters for normal and cancer breast tissues. The results show that the normal tissue has clearly different mean values of scattering, absorption and fluorescence parameters than the cancer tissue, and statistical t-test analysis shows that these differences are significant for all parameters at the 99% confidential level (Table 7). Specifically, higher values of A, C, β-carotene and NADH parameters are found in the normal breast tissue, whereas higher values of B, Hb and collagen parameters are found in the breast cancer tissue. The higher values of the C parameter are consistent with the increased size of tumor cell nuclei, and as a result nucleus-to-cytoplasmic ratio, characteristic of breast cancer (both invasive ductal carcinoma and ductal carcinoma in situ). The higher values of the Hb and collagen parameters are also consistent with the presence of angiogenesis and stromal fibrosis, respectively which are typically seen in breast cancer.

TABLE 7
T-test of the null hypothesis that means of normal and
cancer DRS/IFS parameters are not equal

| Fitting | | | Confidence | tstat | Degree of | Pooled |
|---|---|---|---|---|---|---|
| Parameter | h (99%) | p Value | Interval | Value | Freedom | Sample SD |
| A | 1 | 0 | [0.1872-0.2250] | 28.1151 | 11496 | 0.3882 |
| B | 1 | 0 | [−0.5644-(−0.5201)] | −63.0668 | 8933 | 0.4062 |
| C | 1 | 0 | [0.1453-0.1686] | 44.3469 | 9623 | 0.1662 |
| Hb | 1 | 0 | [−1.1695-(−0.7850)] | −19.7833 | 11368 | 2.597 |
| β-carotene | 1 | 0 | [0.0004-0.0017] | 3.9941 | 10795 | 0.0129 |
| Collagen | 1 | 0 | [−23.4554-(−19.79)] | −30.3962 | 9845 | 34.4264 |
| NADH | 1 | 0.0079 | [0.0281-1.8752] | 2.6558 | 3462 | 8.695 |

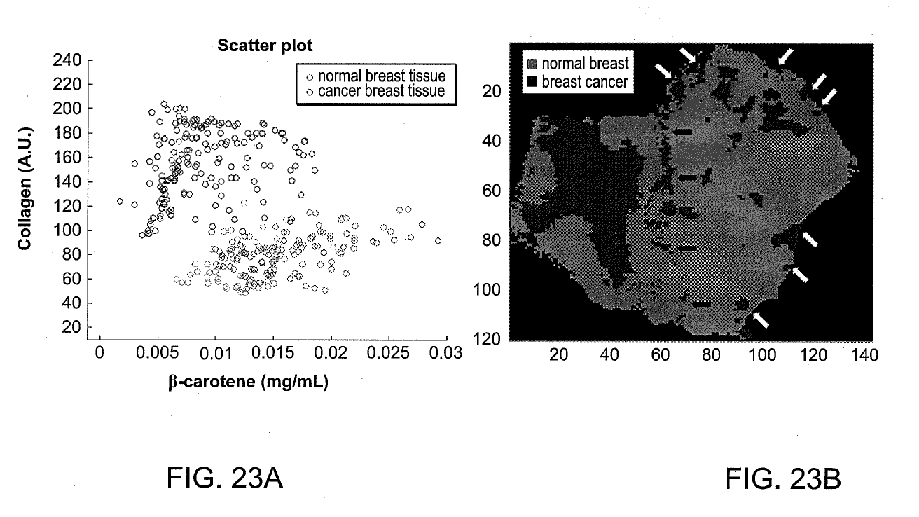

As with the DRS and IFS spectral intensity maps, the individual DRS and IFS parameter maps do not by themselves reliably distinguish normal breast tissue from breast cancer (FIG. 7C-7H). However, normal breast tissue can be reliably distinguished from breast cancer in the tissue scanner images by employing a decision algorithm based on a combination of DRS and IFS parameters. In this method, the IFS collagen and DRS β-carotene parameters are used to distinguish normal breast tissue from all breast lesions including fibrocystic change, fibroadenoma and cancer. Since this measurement includes only normal and cancerous breast tissue, this same process provides adequate diagnostic discrimination, if it is transferable (i.e. is robust). The parameter scatter plot and diagnostic map in FIGS. 23A and 23B show that the DRS-IFS algorithm is transferable and is sufficient to distinguish the tissue types. Representative data points were randomly selected from the two tissue regions for the parameter scatter plot shown in FIG. 23A. The diagnostic map in FIG. 23B identified not only the large focus of invasive ductal carcinoma in the grossly cancerous breast tissue (on the left), but also small (<1 to 3 mm) foci of ductal carcinoma in situ in the grossly normal breast tissue. This indicates that the tissue scanner has sufficient spatial resolution and spectral contrast to detect small foci of cancer in surgical margins.

Thus, a portable, quantitative, multimodal, optical fiber probe-based spectroscopic tissue scanner provide a wide field of view, high resolution and quantitative hyperspectral imaging capability. This exemplifies using a resolution target, tissue-simulating phantoms, animal and human breast cancer tissue. These measurements show that the tissue scanner has sufficient spatial resolution and spectral contrast to detect small foci of cancer in surgical margins.

The tissue scanner enables quantitative spectroscopic measurements without direct contact with the tissue (thereby enhancing its reproducibility), while maintaining a fixed tissue-probe geometry (which reduces extraneous variance from being incorporated in the measurement). It is a simpler design than those of reflectance and fluorescence imaging systems that utilize a complex array of multi-channel optical fiber probes or an operating microscope for signal collection or utilize a high resolution confocal microscope or micro-endoscope to achieve high spatial resolution. Finally, the tissue scanner combines DRS, IFS and Raman with spectral modeling to provide a more complete, quantitative assessment of the metabolic, biochemical and morphological state of tissue and, as a result, a more robust tissue diagnosis than can be achieved with either modality alone or by using purely statistical algorithms for disease diagnosis.

The system provides coupling of multiple collection fibers to the spectrograph system to amplify the acquired signal and improve imaging speed. Further, chemometrics and machine learning algorithms can be used to provide real time processing of the spectral data. While tissue scanner provide powerful hyperspectral capabilities with spectral resolution of ˜2 nm, such resolution may not be necessary for all cancer diagnostics applications.

Non invasive optical techniques, such as diffuse reflectance spectroscopy (DRS), have been used for quantitative tissue characterization and disease diagnosis as described herein. Specifically, DRS provides an assessment of scattering of a sample (which is primarily a function of density and scattering cross sections of internal structures) as well as absorber composition (hemoglobin and beta-carotene).

One of the major challenges of such diagnosis is the accurate and robust analysis of the spectral data, namely the extraction of the absorber concentration and scatterer information, over a variety of sample optical properties and excitation-collection (optical probe) geometries. To this end, diffusion-approximation based solutions of the radiative transport equations and their variants have been employed due to their analytical approach and simplicity of application. However, these methods are not well suited for application in tissues where absorption is high in comparison to scattering (e.g. in cancer angiogenesis) and where the source detector-separation is small. To overcome these difficulties, certain computational techniques such as inverse Monte Carlo models and higher-order analytic approximations have been applied to analyze data.

A look-up table (LUT) approach based on measurements from physical tissue models for optical property determination even at high absorption levels. The primary advantage of the LUT approach is that it does not require modeling of the probe-sample geometry (which is a major challenge for the aforementioned analytical and computational approaches). It also does not necessitate the alteration of the optical probes or measurement at multiple source-detector separation distances. However, the LUT approach involves iterative fitting of the spectra using a non-linear optimization routine, which is computationally expensive (the typical fit time is of the order of a few seconds). This is particularly problematic when the process is deployed in a spectral imaging platform, where data may need to be routinely acquired and processed from a few thousand points. However, the speed of the prediction process is critical to the success of imaging platform applications including the investigation of full tumor margins in intra-operative cancer assessment.

Preferred embodiments of the present invention utilize optical property determination approach based on a non-linear multivariate calibration (MVC) framework. Specifically, a least-squares support vector machine (LS-SVM) can be used to provide a rapid and accurate diagnostic information from diffuse reflectance spectra. Previously, empirical models using multivariate calibration (MVC) schemes, including partial least squares (PLS) and neural networks have been employed for analysis of reflectance spectra. However, PLS does not provide accurate predictions as it is unable to account for the intrinsic nonlinear relationships in light transport in tissue. Furthermore, the present LS-SVM system, which provides a class of kernel machines for non-linear regression, results in a unique and reproducible global solution in contrast to neural networks. Using reflectance spectra collected from physical tissue models over a wide range of optical properties, the prediction accuracy and computation time of LS-SVM is compared with that of PLS and the LUT-approach. Here, PLS is chosen for comparison because of its similarity in approach (MVC) with the present method and fast computation time, whereas LUT is selected for its superior prediction performance. These embodiments have the dual advantage of speed and accuracy of the LS-SVM approach makes it suitable for applications in tissue imaging platforms.

Similar to conventional linear regression modeling, LS-SVM estimation between regressors (x) and the dependent variable (y) can be expressed as:

y=w^{T}x+b (1)

in which w is the regression coefficient and b represents the bias. Analogous to the ridge regression solution of other problems, LS-SVM constructs a trade-off in the objective function (Q_{LS-SVM}) between the regression error (e) and the minimization of the regression coefficients for N training samples (to enhance model robustness):

$${Q}_{LS \text{-} SVM} = \frac{1}{2} {‖ w ‖}^{2} + γ {\sum }_{i = 1}^{N} {e}_{i}^{2} \\ {e}_{i} = {y}_{i} - {w}^{T} {x}_{i} - b \qquad ( 2 )$$

where the relative weight of the two penalties is mediated by the regularization parameter, γ, and ∥w∥ represents the 2-norm of the vector of regression coefficients. The critical step in LS-SVM is the subsequent formulation of a Lagrangian function by incorporating the regression error using Lagrangian multipliers (αᵢ). Solving the resultant Lagrangian problem, it can be shown that the regression problem reduces to the following:

$$y = {\sum }_{i = 1}^{N} {α}_{i} 〈 {x}_{i} , x 〉 + b where {α}_{i} = {( 〈 {x}_{i} , {x}_{i} 〉 + {( 2 γ )}^{- 1} )}^{- 1} ( {y}_{i} - b ) \qquad ( 3 )$$

Here, the notation <•,•> denotes the inner product of the vectors. In essence, the Lagrangian multipliers mediate the relative importance of the individual training samples to the overall model and subsequent prediction. This linear formulation can be readily extended to solve non-linear problems if the inner product is replaced with appropriate kernel functions (K) that satisfy Mercer's conditions. The problem is then modified to:

$$y = {\sum }_{i = 1}^{N} {α}_{i} K 〈 {x}_{i} , x 〉 + b \qquad ( 4 )$$

A polynomial function or a radial basis function can be used for LS-SVM based regression. Clearly, the application of such a function allows higher-dimensional modeling, where the dependent variable estimation of the prediction sample is dependent on the distance of its corresponding regressor from that of the calibration samples in the database.

The kernel functions are parameterized allowing for adjustments with respect to the training data, i.e. the kernel parameter(s) and γ are hyper-parameters specific to the problem dataset. It is worth mentioning that implementation of LS-SVM requires the solution of linear equations as opposed to quadratic optimization for SVM, which makes the former implementation faster. Furthermore, only two parameters need to be optimized for LS-SVM in contrast to three for classical SVM regression, thereby reducing the probability of overfitting the calibration dataset.

For the present embodiment, the diffuse reflectance spectra are input as the regressors (x) to the model whereas the scattering and absorption coefficients are treated as dependent variables (y). Specifically, two models are constructed to predict the scattering and absorption coefficients, respectively, as detailed below.

For characterizing the effectiveness of the LS-SVM approach in extracting the optical properties, diffuse reflectance datasets are employed. A reflectance system was used to acquire steady-state spectra (350-700 nm) from a set of 24 physical tissue models (tissue phantoms) with varying absorption (μₐ, 0.5 to 3.79 mm⁻¹) and reduced scattering (μₛ′, 0.44 to 6.74 mm⁻¹) coefficients. A pulsed xenon flash lamp was used as the excitation source and an imaging spectrograph-CCD combination was used for spectral acquisition. A six-around-one fiber-optic probe was used for delivery and collection of light by placing it in close proximity (˜300 μm) of the samples. The tissue phantoms were fabricated by adding 1 μm polystyrene spheres and India ink to water solutions to simulate typical absorption and scattering properties of biological tissue. Three spectral acquisitions from each sample were averaged for enhanced SNR prior to off-line spectral processing for background correction and normalization using a reflectance standard. The spectral resolution of the system was observed to be approximately 0.78 nm.

The measured dataset consisted of 24 samples in all, was split into calibration and prediction. A leave-one-out protocol was employed to estimate the average prediction error from the 24 samples. For each such splitting, LUT, PLS and LS-SVM models are first generated on the calibration set, by optimizing the model parameters. The optimization of each of these models is performed by inner cross-validation within the calibration dataset. The calibration models are then used prospectively on the prediction data point to determine the absorption (μₐ) and reduced scattering (μₛ′) coefficients. In one embodiment it is assumed that the functional form for the reduced scattering coefficient can be: μₛ′(λ)=μₛ′(λ₀)·(λ/λ₀)^{-B}, where λ₀ is a reference wavelength (e.g. 630 nm) and B represents the wavelength dependence of the scattering coefficient. Since the same scatterers were used in the tissue phantoms, the B parameter (which depends on the shape/size and relative refractive index of the scatterers) remains constant for all samples. Additionally, for the dataset, the computation of the absorption coefficient is identical to that of the India ink concentration due to the lack of other significant absorbers. Thus, these computations involved the extraction of the following two values, namely μₛ′(λ₀) and absorber concentration (which can be directly translated to the absorption spectra by multiplying the intrinsic absorption spectrum of India ink). Note that the B parameter can be readily determined from the computation of the scattering coefficient at a wavelength different from λ₀ using an additional model.

To form the LUT model, the diffuse reflectance values (at all wavelengths) are mapped onto a two-dimensional space constituted by the optical properties at the corresponding wavelengths. Prediction is then performed by executing a non-linear optimization routine which minimizes the error in fitting the sample spectra onto the calibration LUT space (inverse mapping). In contrast to the LUT approach, which creates a single coupled model of the relevant parameters (reflectance values, absorption and scattering coefficients), for the PLS and SVM calculations two separate models for scattering and absorption are constructed. For the PLS regression analysis, the regression vectors for prediction are constructed by employing the number of loading vectors which corresponds to the minimum error in inner cross-validation in the calibration dataset. In these cases, the number of factors is observed to vary between 4 and 7, which ensures that the rank of the calibration model is more than three times smaller than the size of the calibration dataset. The LS-SVM computations are performed using a LS-SVM MATLAB toolbox. Here, a RBF kernel with a typical Gaussian profile (K(xᵢ,xⱼ)=exp(−∥xᵢ−xⱼ∥²/2σ²)) is used for non-linear regression. As the reflectance spectra can be previously normalized using a reflectance standard, further intensity scaling is not required. Note that the normalization step is important to the reduction of skewed behavior of specific pixel intensity values that reside in greater numeric ranges over those having smaller numeric values. The optimal model parameters γ and σ² that provide the smallest error in inner cross-validation are determined by performing a grid search over the range of 1 to 10000 (γ) and 0.01-10 (σ²), respectively.

In addition to the prediction errors, the average computation time for the prediction step is noted for comparison of LUT, PLS and LS-SVM methods. These computations can be performed on an Intel Celeron® CPU 2.00 GHz and 3 GB RAM computer, for example.

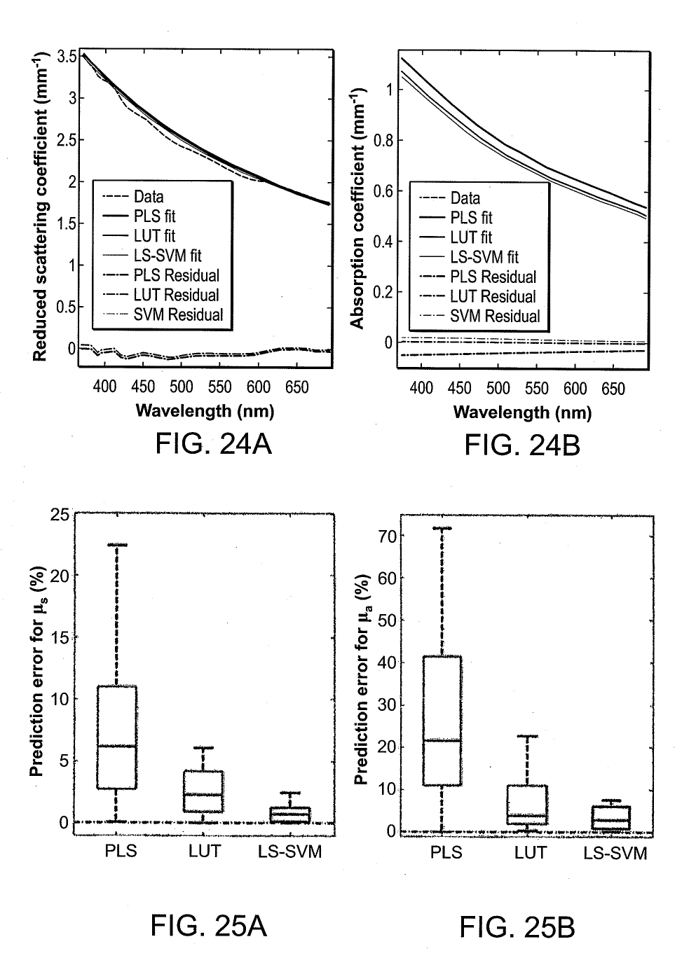

An objective of the LS-SVM approach to measure optical properties, especially in highly attenuating biological media. The scattering and absorption coefficients and the corresponding fits (using all the three modeling approaches) from a representative tissue phantom are shown in FIGS. 24A and 24B. From these figures, one can observe that all the three approaches are valid and can achieve a high level of accuracy. This is also evidenced by the absence of significant features in the accompanying residual plots. However, it is also clear that compared to LUT and LS-SVM based system, PLS provides inferior results, especially for absorption.

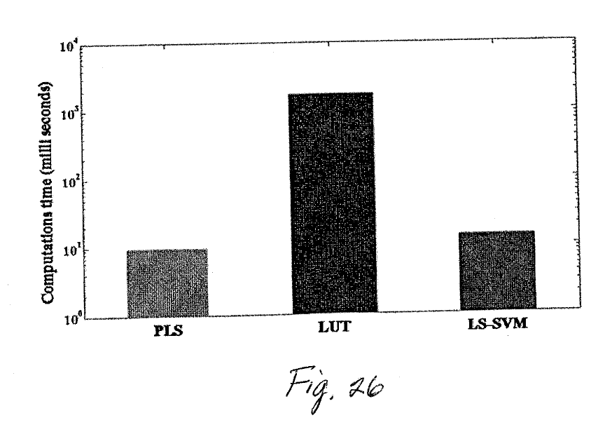

To evaluate these results, a leave-one-out validation method, can be used to obtain an average estimate of the prediction errors over the range of measured optical properties. Note that the limited number of samples of tissue phantoms does not allow for the formation of a complete prospective prediction set. Nevertheless, the presence of an outer loop for validation of the calibration models provides a measure of the ability of the model(s) to generalize relationship between the spectra and optical properties beyond the specific data of the calibration set. FIGS. 25A and 25B show the box plot results of validation error percentage values obtained for the scattering and absorption coefficients. The percentage error (|actual−predicted|/actual*100) is completed instead of the absolute errors due to the large variations in absolute values of these parameters. The mean prediction error percentages for the reduced scattering coefficient are 9.73%, 2.57% and 0.79% for the PLS, LUT and LS-SVM models, respectively. Similarly, the mean prediction error percentages for absorption coefficients are 29.03%, 7.94% and 3.77% for the PLS, LUT and LS-SVM models respectively. Both the approaches substantially improve prediction accuracy as compared to PLS modeling. Furthermore, there is also a corresponding reduction in the standard deviation of the error percentages for both scattering and absorption parameters on application of LS-SVM regression.

These results indicate that PLS is inadequate for modeling of diffuse reflectance spectral data sets, especially in highly attenuating media. This is not surprising given the intrinsic nonlinear relationship between diffuse reflectance and absorption and scattering properties, even under assumptions of semi-infinite media. The specific geometry of the probes, including the source detector separation distance, adds another layer of complexity which necessitates the consideration of nonlinear approaches. Note that curved effects can be modeled by linear multivariate calibration methods such as PLS, but these necessitate the retention of a larger number of factors, with the subsequent risk of inclusion of irrelevant sources of variance in the model. In contrast, LS-SVM based regression is able to address the presence of curved effects, similar to that observed for Raman spectra acquired in turbid media.

Importantly, note the statistically significant improvements in prediction accuracy on application of LS-SVM over the LUT model for both reduced scattering (p<10⁻⁵) and absorption (p=0.028) coefficients. This can be attributed to the ability of support vector machines to distinguish between important and irrelevant training samples by assigning corresponding weights (Lagrangian multipliers) to them. This provides the necessary flexibility to deal with outliers, which unduly influence least squares methods (an integral part of the nonlinear fitting routine in the LUT prediction step). The small prediction errors of the LS-SVM model can be largely attributed to the uncertainty in optical property estimation (based on the gold standard measurement) in the calibration samples and the imperfect construction of the tissue phantoms.

A further objective is to examine the computation time taken by the models to predict the tissue optical properties. FIG. 26 gives the computation time in milliseconds for the three modeling approaches. Clearly, the time taken by the LUT approach is greater than that of PLS and LS-SVM models by more than two orders of magnitude. Although appropriate utilization of the memory cache can reduce the computation time of the LUT approach, nevertheless this remains a major challenge for imaging of large sections of biological tissue. Additionally, in contrast to the PLS and LS-SVM methods, there is an inherent tradeoff between the prediction accuracy and the computation time of the prediction step in the LUT approach. Specifically, construction of a larger LUT (i.e. with finer resolution) provides improved prediction accuracy at the cost of increased computation time.

Note that PLS is marginally faster than LS-SVM based regression in predicting tissue optical properties but this advantage is heavily outweighed by its poorer prediction performance. Thus, LS-SVM provides an improved combination of accuracy and computation time for the determination of tissue optical properties. SVM can also be used in the analysis of Raman spectra as described in detail in U.S. application Ser. No. 13/167,445 filed on Jun. 23, 2011 and in International Application PCT/US2011/046750 filed on Aug. 5, 2011, the entire contents of the above applications being incorporated herein by reference. Thus, SVM can be used in a plurality of light detection modes to improve diagnostic accuracy.

The LS-SVM system provides a method for rapid and accurate determination of tissue optical properties. Measurements indicate that the LS-SVM regression models are able to maintain a high degree of prediction accuracy over a wide range of tissue properties, which is particularly advantageous for the characterization of highly absorbing tissue, a surrogate marker for tumor growth. This method can be used for the estimation of tissue optical properties without further modification. Given the necessity for faster algorithms for tissue imaging applications, preferred embodiments can be extensively employed in histo-pathological assessment including the detection of tumor margins in breast cancer, cervical pre-cancer diagnosis and other cancers including skin, brain or gastrointestinal tract. This system is adaptable to a wide array of tissue properties and probe geometries. Preferred embodiments can be used for the direct determination of absorber concentration, without a priori knowledge of the presence of other absorbers, in applications where the concentration of the analyte of interest (specific absorber) is known (implicit calibration).

While the present invention has been described here in conjunction with a preferred embodiment, a person with ordinary skill in the art, after reading the foregoing specification, can effect changes, substitutions of equivalents and other types of alterations to the system and method that are set forth herein. Each embodiment described above can also have included or incorporated therewith such variations as disclosed in regard to any or all of the other embodiments. Thus, it is intended that protection granted by Letters Patent hereon be limited in breadth only by definitions contained in the appended claims and any equivalents thereof.

What is claimed is:

1. A system for measuring a region of tissue comprising:
a probe for scanning tissue including a fiber optic light delivery and collection system;
a light source system optically coupled to the probe, the system including at least a first light source for diffuse reflectance and a second light source for fluorescence;
a detector system that is coupled to the fiber optic light collection system to detect reflectance and autofluorescence from a tissue sample; and
a scanning system to provide relative movement between the probe and a tissue sample.

2. The system of claim 1 further comprising a third light source for Raman excitation.

3. The system of claim 1 wherein the probe is mounted on the scanning system to translate the probe relative to the region of tissue of a tissue sample.

4. The system of claim 1 wherein the scanning system provides a scanning region that is at least an area of 4 cm².

5. The system of claim 1 wherein the detector system comprises a first detector that detects reflected light and a second detector that detects fluorescence light.

6. The system of claim 5 further comprising a third detector that detects Raman shifted light.

7. The system of claim 1 wherein the scanning system provides a scanning region of at least 10 cm² in area with a scanning time of less than 20 minutes.

8. The system of claim 1 further comprising a data processor that analyzes spectral data.

9. The system of claim 8 wherein the data processor performs margin assessment.

10. A method of measuring tissue comprising:
positioning a probe relative to a region of tissue to be measured, the probe having a light delivery optical fiber and a light collection optical fiber;
illuminating the region of tissue with light from a first light source and a second light source, the light being delivered through the light delivery optically fiber;
collecting light returning from the region of tissue with the light collection optical fibers, a first collection optical collecting reflected light and a second light collection optical fiber collecting fluorescence light; and
detecting reflected and fluorescence light collected from the tissue at a plurality of positions.

11. The method of claim 10 wherein the tissue comprises excised breast tissue.

12. The method of claim 10 wherein the tissue comprises skin tissue.

13. The method of claim 10 further comprising detecting Raman scattered light.

14. The method of claim 10 further positioning a tissue sample on an optically transmissive material and transmitting excitation light through the material in a scan pattern.

15. The method of claim 10 further comprising analyzing spectral data to perform margin assessment of the tissue.

16. The method of claim 10 further comprising analyzing the detected light with a data processor.

17. The method of claim 16 wherein the analyzing step further comprises determining whether the tissue is cancerous or normal tissue.

18. The method of claim 16 wherein the analyzing step comprises using a nonlinear representation to process spectral data.

19. The method of claim 18 further comprising using a support vector machine to process spectral data.

20. The method of claim 18 further comprising determining whether the region of tissue includes cancerous tissue.

21. The method of claim 10 further comprising actuating relative movement between a fiber optic probe and a tissue sample using a scanning system.

22. The method of claim 21 further comprising selecting scan parameters including scan area resolution and scan time.

23. The method of claim 22 further comprising collecting spectral data from a scan area using a single light spot size.

24. The method of claim 10 wherein diffuse reflectance light is detected by a first detector and fluorescence light is detected by a second detector.

25. The method of claim 24 wherein Raman light is collected by a third collection fiber and detected by a third detector.
